(19) United States
(12) Patent Application Publication
(10) Pub. No.: US 2006/0263767 A1
(43) Pub. Date: Nov. 23, 2006

(54) Ultrasensitive detection of prions by automated protein misfolding cyclic amplification

(75) Inventors: Joaquin Castilla Castrillon, Galveston, TX (US); Paula Saa Prieto, Galveston, TX (US); Claudio Soto-Jara, Friendswood, TX (US)

Correspondence Address: FULBRIGHT & JAWORSKI L.L.P., 600 CONGRESS AVE., SUITE 2400, AUSTIN, TX, 78701, US

(73) Assignee: THE BOARD OF REGENTS OF THE UNIVERSITY OF TEXAS SYSTEM

(21) Appl. No.: 11/407,690

(22) Filed: Apr. 20, 2006

Related U.S. Application Data
(60) Provisional application No. 60/673,302, filed on Apr. 20, 2005.

(51) Int. Cl. C12Q 1/70; C12Q 1/68

(52) U.S. Cl. 435/5; 435/6

(57) ABSTRACT

A highly sensitive method is provided for the detection of prions in a sample. These methods may be used to diagnose prion mediated transmissible spongiform encephalopathies such as bovine spongiform encephalopathy, Creutzfeldt-Jakob disease, scrapie, or chronic wasting disease. In particular a method for serial automated cyclic amplification of prion is disclosed. The method is both rapid and highly sensitive making it ideal for high throughput testing.

This application claims priority to U.S. Provisional Patent application Ser. No. 60/673,302 filed Apr. 20, 2005, which is incorporated by reference in its entirety.

The United States government may own certain rights to this invention pursuant to grant number AG024642-01 from the National Institutes of Health.

### BACKGROUND OF THE INVENTION

I Field of the Invention

The present invention relates generally to pathology, biochemistry, and cell biology. In particular the invention provides methods, compositions, and apparatuses for the detection of infectious proteins or prions in samples, including the diagnosis of prion related diseases.

II. Description of Related Art

Prion diseases, which are also called transmissible spongiform encephalopathies (TSEs), comprise a group of fatal infectious neurodegenerative diseases that include Creutzfeldt-Jakob disease (CJD), kuru, Gerstmann-Sträussler Sheinker syndrome (GSS), fatal familial insomnia (FFI) and sporadic fatal insomnia (sFI) in humans and scrapie, bovine spongiform encephalopathy (BSE) and chronic wasting disease (CWD) in animals (Collinge, 2001; Prusiner, 2001). These diseases are characterized by brain vacuolation, astroglyosis, neuronal apoptosis and the accumulation of the misfolded prion protein (PrP^{Sc}) in the central nervous system (Prusiner, 1998).

The hallmark event of prion disease is the formation of an abnormally folded protein called PrP^{Sc}, which is a post-translationally modified version of a normal protein, termed PrP^{C} (Cohen and Prusiner, 1998). Chemical differences have not been detected to distinguish both PrP isoforms (Stahl et al., 1993) and the conversion seems to involve a conformational change whereby the α-helical content of the normal protein diminishes and the amount of β-sheet increases (Pan et al., 1993). The structural changes are followed by alterations in the biochemical properties: PrP^{C} is soluble in non-denaturing detergents, PrP^{Sc} is insoluble; PrP^{C} is readily digested by proteases, while PrP^{Sc} is partially resistant, resulting in the formation of a N-terminally truncated fragment (Baldwin et al., 1995; Cohen and Prusiner, 1998). See table I for the nomenclature used to refer to different species of PrP.

TABLE 1
PrP, refers to the total prion protein without making a distinction for
different isoforms
PrP^{C}, normal, non-pathogenic, non-infectious cellular protein present
in healthy people. This form is rich in α-helical conformation,
is soluble and protease-sensitive.
PrP^{Sc}, disease-associated misfolded prion protein present in
individuals affected by TSE. This form is infectious, rich in β-sheet
conformation, insoluble and mostly protease-resistant.
PrP^{res}, refers to a β-sheet rich, protease-resistant prion protein,
which may or may not be identical to PrP^{Sc}. In particular, this name
is used to refer to in vitro produced protease resistant protein which
has not been experimentally shown to be infectious.
PrP27-30, correspond to the protein core that remains resistant
after protease treatment of
PrP^{Sc} or PrP^{res}. It consists of the last two thirds of the protein.

At present there is no accurate premortem diagnosis for TSEs (Brown et al., 2001; Collins et al., 2000; Ingrosso et al., 2002; Soto, 2004). For human diseases, diagnosis is based mainly on clinical examination and the disease is considered possible, or probable, depending upon the degree to which the clinical symptoms fit the standard guidelines. Definitive diagnosis can only be made postmortem by brain histological analysis (Ingrosso et al., 2002; Kordek, 2000) of spongiform changes, astrogliosis and amyloid plaques (although these plaques are not consistently seen in all TSEs). Although brain biopsy has been used to establish a definitive diagnosis, it is strongly discouraged because it is invasive and costly. Moreover, a brain biopsy sometimes produces a false-negative result, because the tissue sample has been taken from an unaffected area of the brain.

The serious consequences of BSE epidemics motivated the European Community to implement a system to evaluate and validate biochemical tests aimed at rapidly detecting infected animals (Bird, 2003; Butler, 1998). Postmortem identification of sick cattle by histological analysis of the brain is accurate (Heim and Wilesmith, 2000). However, this procedure is time consuming, labor intensive and cannot be carried out on a large scale. New tests were developed to enable processing multiple samples in just a few hours, so that commercialization of the animals could be withheld until results were available. Two campaigns were undertaken to evaluate 9 different tests using blind samples from BSE-infected and normal cattle (Editorial, 2001; Bird, 2003; Moynagh and Schimmel, 1999). Using sensitivity and specificity as criteria, 5 tests were approved by the European Community for BSE detection. All 5 tests are based on immunodetection of the pathological PrP^{Sc} isoform and 4 use proteolysis to distinguish PrP^{C} from misfolded PrP. However, the current sensitivity of these test enable detection of prions only in the brain at (or close to) the symptomatic phase of the disease.

Strikingly, a recent study reported the first possible case of vCJD contracted through blood transfusion (Llewelyn et al., 2004). A 69-year-old person developed vCJD symptoms 6.5 years after receiving a transfusion of red cells donated by an individual that developed symptoms of vCJD 3.5 years after donating blood. If the possibility of transmission of vCJD by blood transfusion is supported by the discovery of similar cases, it could lead to dramatic consequences for a potential vCJD epidemic, because it would indicate that blood harbors infectivity several years before the onset of clinical symptoms. Because of this possibility the development of a sensitive and presymptomatic blood test for CJD is a top priority. Additional evidence for blood transfusion as a possible route of TSE transmission comes from the elegant experiments by Houston and Hunter, in which transmission of BSE occurred through blood transfusion in sheep (Houston et al., 2000; Hunter et al., 2002). Interestingly, in these experiments the blood used for transfusion was obtained from sheep midway through the incubation period. Infectivity has also been shown in blood during the incubation period and symptomatic phase in a rodent model of vCJD (Brown et al., 1999). These findings have important implications not only for TSE diagnosis but also for several other applications, such as blood bank safety and plasma products industry.

Formation of PrP^{Sc} is not only the most likely cause of the disease, but also is the best known marker. Detection of PrP^{Sc} in tissues and cells correlates widely with the disease and with the presence of TSE infectivity. Treatment that inactivates or eliminates TSE infectivity also eliminates PrP^{Sc} (Prusiner, 1991). The identification of PrP^{Sc} on human or animal tissues is considered key for TSE diagnosis (WHO Report, 1998). One important limitation to this approach is the sensitivity, since the amounts of PrP^{Sc} are high (enough for detection with conventional methods) only in the CNS at the late stages of the disease. However, it has been demonstrated that at earlier stages of the disease there is a generalized distribution of PrP^{Sc} (in low amounts), especially in the lymphoreticular system (Aguzzi, 1997). Indeed, it has been reported the presence of PrP^{Sc} in palatine tonsillar tissue and appendix obtained from patients with vCJD (Hill et al., 1997). Although it is not known how early in the disease course tonsillar or appendix biopsy could be used in vCJD diagnosis, it has been shown that in sheep genetically susceptible to scrapie, PrP^{Sc} could be detected in tonsillar tissue presymptomatically and early in the incubation period. However, PrP^{Sc} has not been detected in these tissues so far in any cases of sporadic CJD or GSS (Kawashima et al., 1997). The normal protein is expressed in white blood cells and platelets and therefore it is possible that some blood cells may contain PrP^{Sc} in affected individuals (Aguzzi, 1997). This raises the possibility of a blood test for CJD, but this would require an assay with a much greater degree of sensitivity than those currently available.

One method that can consistently and reproducibly detect prions in blood is the infectivity bioassay (Brown et al., 1998; Brown et al., 2001; Ingrosso et al., 2002). However, bioassays are limited for widespread use by the length of time that it takes to obtain results (several months to years) and the species barrier effect, but these experiments enable to estimate that the concentration of PrP^{Sc} in buffy coat is between 1×10⁻¹⁴ M and 1×10⁻¹⁶ M (i.e., 60,000-6,000,000 molecules of PrP^{Sc} per ml of buffy coat) (Brown et al., 2001; Soto, 2004).

Recently a more rapid prion detection method was developed based on the ability of prions to replicate in vitro in cell lysates containing PrP^{C}. This techniques termed protein misfolding cyclic amplification (PMCA) involved mixing samples with a “non-pathogenic conformer”, incubating the mixture, disaggregating proteins, then performing repeated incubation and desegregation steps, see WO 0204954; Saborio et al., 2001; Castilla et al., 2004 and Saa et al., 2004, all of which are hereby incorporated by reference. In vitro amplified prion was then detectable with a high level of sensitivity that is typically achieved by Western blot or ELISA assays. This technique offered the significant advantage of rapid results, however, still was not believed as sensitive as the infectivity bioassay. Additionally though prion could be amplified from blood using this assay, it is desirable to improve the level of sensitivity and/or increase the reproducibility of the assay, especially for diagnostic purposes. Subsequent modification of this assay by other researchers has shown that prion can be continually replicated; however, improvements in the level of replication are still needed for diagnostic tests (Bieschke et al. 2004). Thus, currently there continues to be a need for a rapid method for the detection of prion that is sensitive enough to detect low level prion contamination.

### SUMMARY OF THE INVENTION

The present invention provides a highly sensitive method for detecting prion in a sample, termed “serial automated protein misfolding cyclic amplification” (saPMCA). The term “prion” as used herein is defined as an infectious protein consistent with its usage in the prior art. Specifically a prion has the ability to alter the conformation of a homologous protein such that the homologous protein, in its altered conformation, has substantially the same activity as the original prion.

Some methods of the invention involve amplification of prion protein by saPMCA that enables high sensitivity detection of prion in a sample. In certain embodiments the method for detecting prion involves amplification of the prion, serial amplification of the prion, detection of prion and inactivation of residual infectious prion protein. The methods may involve one or more of steps (a), (b), (c), (d) and (e) below:
(a) Mixing a sample with non-pathogenic protein to make a reaction mixture;
(b) A primary amplification step comprising:
   (i) incubating the reaction mix,
   (ii) disrupting the reaction mix, 25644548.1 6
   (iii) repeating steps (b)(i) and (b)(ii) one or more times
(c) Performing serial amplification comprising:
   (i) removing a portion of the reaction mix and incubating it with additional non-pathogenic protein,
   (ii) repeating amplification steps (b), (iii) repeating steps (c)(i) and (c)(ii) one or more times;
(d) Detecting prion in the serially amplified reaction mix;
(e) Inactivating residual prion.

Each step is further described below:

(a) Mixing a sample with non-pathogenic protein to make a reaction mix. The term “sample” refers to any composition of matter capable of being contaminated with prion. For example a sample may comprise a tissue sample from an animal suspected of having a TSE. The term “non-pathogenic protein” as used herein refers to protein that is homologous in amino acid sequence to a prion and is capable of being converted into a prion. Thus, “the reaction mix” refers to a composition minimally comprising a sample and non-pathogenic protein. In some embodiments, the reaction mix further comprises a “conversion buffer” that is favorable for prion replication. An exemplary conversion buffer may comprise 1× phosphate buffered saline (PBS) with 150 mM additional NaCl, 0.5% TritonX-100 and a protease inhibitor cocktail.

(b) The primary amplification step involves incubation of the reaction mix under conditions that favor prion replication (b)(i), followed by disruption of the reaction mix in order to break apart protein aggregates (b)(ii). As used herein the term “disrupting” refers to any method by which proteins may be disaggregated. Exemplary disaggregation methods include treatment with solvents, modification of pH, temperature, ionic strength, or by physical method such as sonication or homogenization. These two steps are repeated one or more times thereby amplifying the prion (b)(iii).

(c) The reaction mix from the primary amplification is subjected to serial amplification which greatly enhances prion replication. In this step a portion of the reaction mix is incubated with additional non-pathogenic protein (c)(i) to make a serially amplified reaction mixture. As used herein “additional non-pathogenic protein” may be from the same source as the non-pathogenic protein used in primary amplification (a) or it may be from a different source. In some embodiments serial amplification will comprise repeating the steps of primary amplification (c)(ii) one or more times. In further embodiments the steps of serial amplification (c)(i) and (c)(ii) are repeated one or more times to further amplify prion from the sample (c)(iii). By subjecting the sample to sequential serial amplifications the degree of sensitivity is greatly enhanced, allowing detection of fewer than about 10⁵, 10⁴, 10³, or any range derivable therein or even fewer prions. In certain embodiments this high sensitivity allows for detection of prions with greater sensitivity than the infection bioassay, which has been the gold standard test for the presence of prion.

(d) Prion can be detected in the serially amplified reaction mix by both direct and indirect assays known to those of skill in the art. Exemplary methods for detection of prion in the serially amplified reaction mix are outline below.

(e) Residual prion may be inactivated by various methods known to those in the art, such as treatment with a concentrated base or treatment at high temperature, for example, treatment with 2N NaOH for 1 hour and/or autoclaving at 134° C. for 18 min. This would eliminate the danger of prion as biohazardous waste and also help to minimize contamination that could occur when testing multiple samples. Alternatively, the non-pathogenic PrP substrate can be modified in such a way that after conversion by saPMCA can be easily inactivated by for example adding a proteolytic cleavage site.

The present invention also provides a method to diagnose a disease in an animal by detecting the presence of a prion in a sample from the animal, such as a method comprising one or more of steps (a), (b), (c), (d) and (e) described above. As used herein “animal” refers to any animal that is susceptible to a prion disease. For examples animals include but are not limited to a variety of mammals such as humans, cows, sheep, deer, and elk. Detection of prion in the reaction mix is indicative of a positive diagnosis for a prion disease. As defined herein “a prion disease” is any disease transmissible via a prion vector, such diseases comprising CJD (sCJD, fCJD, iCJD and vCJD), GSS, kuru, FFI, sFI, scrapie, BSE and CWD.

It is contemplated that the detected prion could comprise abnormally folded PrP protein typically termed PrP^{Sc}. For example the prion protein may be mammalian PrP^{Sc}. The PrP^{Sc} could comprise sheep PrP^{Sc}, bovine PrP^{Sc}, mouse PrP^{Sc}, human PrP^{Sc}, deer PrP^{Sc} or a PrP^{Sc} from other mammals. In other embodiments the prion may comprise a yeast prion, for example abnormal conformations of the Ure2 or Sup35 proteins.

It is contemplated that the method of the invention may be used to detect prion in a wide variety of samples. In some embodiments the sample is a tissue sample from an animal. Tissues samples may comprise samples from brain, or from peripheral organs. For examples, samples from spleen, tonsils, or other lymphoid organs may be preferred since it has been shown that they contain relatively high amounts of prion in prion infected animals. Other biological fluids such as cerebrospinal fluid, blood, urine, milk, tears, saliva, may be used. In particular embodiments samples maybe be taken from blood. Detection of prion in blood samples is of great interest since it represents an easily harvested tissue that can be readily taken from a living organism. Thus, the current invention could enable the detection prion diseases from blood samples with a sensitivity sufficient to detect preclinical disease, an important advance in the art.

In certain embodiments of the current invention disruption of protein in the reaction is by sonication. To prevent contamination it is preferred that the sonication apparatus not come in direct contact with the samples. Thus sonication with a commercially available microsociator may be performed. The sonication apparatus may be automated and capable of programmed operation thus allowing high throughput sample amplification. For example sonication could comprise a pulse of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or more seconds of sonication, or any range derivable therein, at 10%, 20%, 30%, 40%, 50%, 60%, 70%, 80%, 90%, or 100% potency, or any range derivable therein. It is also preferable that the reaction mixes be kept in a sealed environment to prevent evaporation. For example amplification may be carried out while samples are maintained in a sealed plexiglass enclosure.

In certain embodiments of the invention the parameters of the sonication step may be varied over the course of amplification. For example the sonication time and/or sonication potency maybe increased or decreased after each cycle. In certain embodiments the sonication parameters (i.e. the time and potency) could be preprogrammed for each step of cyclic amplification.

In certain embodiments of the present invention it is contemplated that incubation of the reaction mixture may be at temperatures at or near physiological temperatures. For example incubation at about 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., to about 50° C., or any range derivable therein. In certain applications of the invention, the incubation is at about 37° C. It is also envisioned that the temperature may be varied. For instance each time the reaction mix is incubated the temperature may be increased or decreased. It is also contemplated that the temperature of the reaction mix could be modified prior to disruption of the reaction mixture. In certain embodiments the temperature of the reaction mixture is monitored and/or controlled by a programmable thermostat. For example the sample may be placed in an automated thermocycler thus allowing the temperature of the reaction mixture to be programmed over the course of amplification.

It is also contemplated that incubation of the reaction mixture could be performed over a range of time periods. For example the reaction mixture may be incubated for about one minute to about 10 hours. In a certain embodiments the incubation time is about or at least about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200 minutes or any range derivable therein. In an even further embodiment, the reaction mix is incubated for about 30 minutes. It is also contemplated that the incubation time may be varied through out the amplification. For example the incubation time may be increased or decreased by an increment of time after each amplification step. In still further embodiments the disruption apparatus is automated such that incubation times may be programmed.

In some embodiments of the current invention incubation and disruption (steps (b)(i) and (b)(ii)) are repeated many times, it is contemplated that they could be repeated at least or at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 times, or any range derivable therein. It is envisioned that in some embodiments of the present invention primary amplification (step (b)) would take place over a period of about three days or less. This may be preferable since in some cases the non-pathogenic protein or other cofactors may have a limited stability and extended incubation may result in an eventual fall-off of the conversion rate. In particular it has been shown that PrP^{C} conversion rates drop after about 75 hours of incubation.

In certain embodiment the invention steps (c)(i) and (c)(ii), serial amplification could be repeated multiple times. For example steps (c)(i) and (c)(ii) could be repeated 1, 2, 3, 4, 5, 6, 7, 8, 9, 10, 11, 12, 13, 14, 15, 16, 17, 18, 19, 20, 21, 22, 23, 24, 25, 26, 27, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 times, or any range derivable therein. In certain embodiments the additional non-pathogenic protein is stored as lyophilized powder or tablets, and/or is kept frozen, to prevent protein degradation, prior to mixing it with the reaction mix or serial reaction mix. In further embodiments the number serial amplification steps may be preprogrammed for automated amplification.

In a certain embodiments of the current invention the reaction mix may further comprise a sample, non-pathogenic protein and a conversion buffer. In some embodiments the conversion buffer comprises a salt solution and detergents. The conversion buffer may further comprise a metal chelator. This is of particular advantage since Cu²⁺ and to some extent Zn²⁺ interferes with the amplification of prion in the case of PrP^{C} to PrP conversion. In a preferred embodiment the metal chelator is EDTA. The reaction mix may also comprise additional elements, for example, one or more buffers, salts, detergents, lipids, protein mixtures, nucleic acids and/or membrane preparations.

In certain embodiments of the current invention the non-pathogenic protein may be from a cell lysate. The cell lysate may comprise a crude cell lysate or a cell lysate that has been treated in such a way as to enrich the lysate for said non-pathogenic protein. The cell lysate may be a liquid, semi-liquid, or a lyophilized protein powder or tablet. In some embodiments the cell lysate comprises a brain homogenate. In some embodiments the brain homogenate is a mammalian brain homogenate. In certain embodiments it may be preferable that the cell lysate be derived from the same species of organism as the test sample. The cell lysate may also be from cells that over express the non-pathogenic protein. In some embodiments the cell lysate is from cells that have been transformed with a nucleic acid expression vector that expresses the non-pathogenic protein. For example the non-pathogenic protein may be from cell lysate of tissue culture cells that over express PrP such as neuroblastoma cells that over express PrP^{C}. Also the non-pathogenic protein can be recombinantly expressed in bacteria.

In certain embodiments of the current invention the non-pathogenic protein may comprise proteins with an amino acid sequence that is homologous to PrP^{C}. For example the non-pathogenic protein may be identical or highly homologous to the PrP protein from mice, humans, cattle, sheep, goat, and/or elk given by GenBank accession numbers NP_{—}035300, NP_{—}898902, AAP84097, AAU02120, AAU02123 and AAU93884 respectively, all incorporated herein by reference. In some embodiments the non-pathogenic protein may comprise a PrP^{C} with an altered amino acid sequence. For example, the non-pathogenic protein may comprise PrP^{C} with amino acid substitutions, deletions or insertions. Some preferred mutations include known mammalian PrP polymorphisms (Table 2). In other cases preferred mutation may be those that have been shown in humans to increase risk of Prion diseases (Table 2). It is envisioned that such mutant proteins may be used to further enhance the sensitivity of the method of the invention. In other embodiments the method of the invention may be used to study the susceptibility of certain mutant PrP proteins to conversion by PrP^{Sc}.

In some embodiments of the current invention the non-pathogenic protein may be from cell that expresses the non-pathogenic protein as a fusion protein. For example the coding sequence for the non-pathogenic protein may be fused to other amino acid coding sequences. For example the fused amino acid coding sequences could comprise coding sequence for a reporter protein, a detectable tag, a tag for protein purification, or a localization signal. Additionally, non-pathogenic protein may be labeled for detection, for example, by incorporation or radioactive amino acids or covalent modification with a fluorophore.

It is also contemplated that the non-pathogenic protein may be modified in such a way as to increase its ability to undergo conversion into prion. In preferred embodiments the non-pathogenic protein may be pretreated to alter glycosylation. This step may further enhance the conversion rate of non-pathogenic protein into prion since it has been previously shown that less glycosylated forms of PrP^{C} are preferentially converted into PrP^{Sc} (Kocisko et al., 1994). For example PrP^{C} may be treated with phospholipase C in order to remove phosphatidylinositol prior to mixing with the sample. Alternatively, recombinant protein can be modified to change the amino acids where glycosylation moieties are attached, so that stably mono- or un-glycosylated forms are synthesized in cells.

In further embodiments of the current invention samples may be treated or fractionated in such a ways as to concentrate the protein of the sample prior to saPMCA. For example protein may be concentrated by phosphotungstic acid (PTA) precipitation, or binding to ligands, shown to interact specifically to PrP^{Sc}, such as conformational antibodies, certain nucleic acids, plasminogen or various short peptides (Soto et al., 2004). It is also contemplated that samples may be fractionated. For example, the fraction that is insoluble in mild detergent could be harvested, a procedure that would increase the concentration of prion within the sample (WO 0204954).

It is contemplated that detection of amplified prion in a reaction mix or serially amplified reaction mix may be via a variety of methods that are well known to those in the art. In one embodiment the reaction mix or serial reaction mix is treated with a protease, such as proteinase K, and then prion is detected by Western blot or by ELISA using anti-prion antibody. In preferred embodiments an anti-PrP antibody may be used, for example the 3F4 monoclonal antibody. In some embodiments the ELISA assay may be a two-site immunometric sandwich ELISA. In other embodiments the prion may be detected by conformational-dependent immunoassay (CDI). It is also contemplated that amplified prion may be detected by animal bioassay, wherein test animal are inoculated with the reaction mix or serial reaction mix and assessed for clinical symptoms. Amplified prion may be also be detected by functional assays, such as by their ability to infect certain mammalian cells in culture (Klohn et al., 2003). Finally, amplified prion may be detected by in-direct methods such as some of the spectroscopic techniques under development, including multispectral ultraviolet fluoroscopy, confocal dual-color fluorescence correlation spectroscopy, fourier-transformed infrared spectroscopy or capillary electrophoresis, and Fluorescence Resonance Energy Transfer (FRET) (Soto et al., 2004).

The current invention also provides an apparatus for amplification and detection of prion protein. The apparatus comprises a programmable microplate sonicator. The microplate sonicator may be programmed for multiple cycles, incubation times, sonication potency and sonication periods. The apparatus may further comprise an incubator capable of being programmed for a range of different incubation temperatures. In certain embodiments the apparatus may also comprise programmable robotic probes for sample and reaction mix manipulation. It is also contemplated that separation and of non-pathogenic protein and prion and detection of prion in the reaction mix may be automated. For example prion may be detected as described herein with automated ELISA methods as described in U.S. Pat. No. 6,562,209 or by automated western blot as described in U.S. Pat. Nos. 5,914,273 and 5,567,595. Wherein the non-pathogenic protein is fluorescently labeled conformational changes may be detected by FRET and monitored “real time” as the sample is subjected to saPMCA.

In some embodiments, the invention relates to a kit for detecting prion in a sample comprising: non-pathogenic protein. In some embodiments, the kit may further comprise: an enclosure for sample amplification such as a microtiter plate, or sample tubes; an amplification buffer that is added to the sample and non-pathogenic protein prior to amplification; positive and negative control samples for saPMCA, wherein the positive control sample contains prion and the negative control sample does not; a decontamination buffer for inactivation of prion, for example an spray, solution or wipe comprising 2N sodium hydroxide; materials for separating prion from non-pathogenic, for instance a proteinase K digestion buffer, or a prion fractionation buffer; materials for detection prion protein, for example PrP specific antibodies for Western blotting or ELISA tests.

As used herein, “sensitivity” refers to the ability of an assay to detect the presence of pathogenic prion conformer (i.e., to give a high percentage of true positive reactions and a low percentage of false negative reactions). As used herein, specificity refers to the ability of an assay to reliably disntinguish between pathogenic conformer and PrP^{C} (i.e., to give a low percentage of false positive reactions and a high percentage of true negative reactions). Aspects of the invention include methods capable of detecting less than 2, 5, 10, 50, 100, 200, 500 attograms (ag), 1, 0.9, 0.8, 0.7, 0.6, 0.5, femtogram (fg) or less of prion in a 10 μl sample. In further aspects, the methods are capable to detecting 3×10⁷, 1×10⁷, 5×10⁶, 1×10⁶, 5×10⁵, 1×10⁵, 5×10⁴, 1×10⁴, 5×10³, 1×10³, 100, 50, 26 molecules of prion or less in a sample (e.g., per 20 μl of sample), including all values in between. In still further aspects, the methods of the invention are capable of detecting prion in sample dilutions of 1×10⁻⁷, 5×10⁻⁷, 1×10⁻⁸, 5×10⁻⁸, 1×10⁻⁹, 5×10⁻⁹, 1×10⁻¹⁰, 5×10⁻¹⁰, 1×⁻¹¹, 5×10⁻¹¹, 1×10⁻¹², 5×⁻¹², or more of 263K scrapie brain, including all values in between. Methods of the invention will typically be capable of a 4×10⁵, 1×10⁶, 5×10⁶, 1×10⁷, 1×10⁸, 1×10⁹, 3×10⁹ or greater fold increase in sensitivity as compared to Western blot analysis, including all values in between. Embodiments of the invention include a specificity of detection greater than 90%, 92%, 95%, 98%, 99% up to 100% of assays caple of distinguishing pathogenic and non-pathogenic prion.

In still further embodiments methods of the invention can be used to detect biological samples potentially contaminated or contaminated with prions. Sample suspected of contamination include, but are not limited to blood (plasma, red cells, platelets, etc), urine, cerebrospinal fluid, and any product derived or isolated from such samples. In certain aspects a suspect sample may be an organ, tissue, or cells of an animal or a human to be used for organ transplant, grafting, or purification of products from such a sample.

Embodiments discussed in the context of a methods and/or composition of the invention may be employed with respect to any other method or composition described in this applications. Thus, an embodiment pertaining to one method or composition may be applied to other methods and compositions of the invention as well.

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawing is part of the present specification and is included to further demonstrate certain aspects of the present invention. The invention may be better understood by reference to the drawing in combination with the detailed description of specific embodiments presented herein.

FIG. 1. A flowchart depicting an exemplary saPMCA procedure. Block arrows indicate steps that may be repeated to generate multiple amplification or serial amplification cycles.

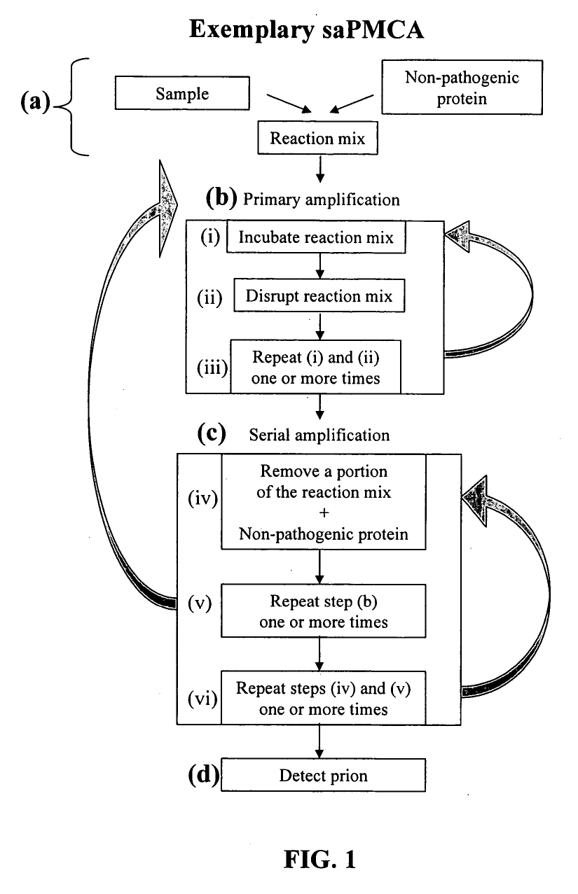

FIG. 2. PrP^{Sc} detection in blood of scrapie infected hamsters by PMCA. Blood samples from groups of scrapie inoculated and control animals were taken at different times during the incubation period. One ml of blood was used to prepare buffy coat as described (Castilla et al., 2005). Samples were subjected to 144 cycles of PMCA. Ten μl of the sample from this first round of amplification were diluted into 901l of normal brain homogenate and a new round of 144 PMCA cycles was performed. This process was repeated for a total of seven times. Each panel represents the results obtained in the 7th round of PMCA with the samples in each group of animals. Ix: samples from hamsters infected with 263K scrapie; Cx: samples from control animals injected with PBS. All samples were treated with PK before electrophoresis, except the normal brain homogenate (NBH) in which -PK is indicated.

FIG. 3. Proportion of PrP^{Sc} blood positive animals at different times during the incubation period. The percentage of samples scoring positive for PrP^{Sc} in blood is represented versus the time after inoculation in which samples were taken. Two phases of PrP^{Sc} detectability were observed: an early stage during the incubation period, which likely corresponds to the time in which peripheral prion replication in lymphoid tissues is occurring and a second phase at the symptomatic stage where the brain contains extensive quantities of PrP^{Sc}.

FIG. 4. Minimum quantity of PrP^{Sc} detected by saPMCA. Aliquots of scrapie hamster brain homogenate were serially diluted into conversion buffer to reach 1×10⁻¹² and 1×10⁻¹⁴ dilutions. Four aliquots of 20 μl of each dilution were mixed in 4 separated tubes with 80 μl of normal brain homogenate and subjected to 144 PMCA cycles. Thereafter, a volume of 20 μl was used for western blotting after PK digestion and 10 μl were diluted into 90 μl of normal brain homogenate and the samples were subjected to a second round of 144 PMCA cycles. The procedure was repeated several times to reach 7 successive rounds of PMCA. S1, S2, S3 and S4 correspond to the four replicated tubes in each dilution. As a negative control, normal brain homogenate diluted 10⁻¹² into conversion buffer was used and subjected to the same scheme of saPMCA. This experiment was also done in 4 replicated tubes and C1, C2, C3 and C4 represent each result.

### DETAILED DESCRIPTION OF THE INVENTION

Disclosed herein is a method to detect prion in a sample; this method can be used to diagnose a variety of diseases in animals. The methods for detection of prion of the invention improve sensitivity and reduce the time necessary for high sensitivity detection of prion in samples. The current invention would enable high throughput, accurate and sensitive screening of samples, as well as diagnosis of clinical disease. For example with a cow, the method could be used to diagnose bovine spongiform encephalopathy (BSE). With sheep the method could also be used to diagnose scrapie. In the cases of deer and elk the methods could be used to diagnose CWD. The advantages of the current invention include testing of live animals for infection to protect against unnecessary culling of herds or inadvertent introduction of prion into the food chain.

It is also contemplated that the diagnostic methods described could be applied to humans and human diseases. Prion diseases that could be diagnosed in humans comprise Creutzfeldt-Jakob disease (CJD), kuru, fatal familial insomnia, Gerstmann-Straussler-Scheinker disease, or sporadic fatal insomnia. Again the method of the invention offers significant advantages over currently available method for diagnosis of these neurologic disorders. For instance the cognitive tests and clinical signs currently used for diagnosis of CJD can only indicate a probable diagnosis. The invention offers an objective method by which positive diagnosis may be made with little chance of false positive or negative results. Additionally the sensitivity of the test enables the detection of disease from peripheral tissues, such as blood, which is would be much less invasive and expensive than current brain biopsy procedures. The invention also provides sensitivity that is high enough such that disease may be detected and diagnosed prior to the onset of clinical symptoms.

Misfolded proteins that mediate other disease states may also be detected via the method of the invention. For example misfolded Aβ also known as beta-amyloid, known to be associated with Alzheimer's disease, may be detected. This method could further be used as a diagnostic test for Alzheimer's disease. As is the case with CJD diagnosis of Alzheimer's is currently based primarily on cognitive tests, and a biochemical testing procedure would be a great advantage.

Another application of the present invention is as a high throughput method of screening for compounds that enhance or inhibit conversion of non-pathogenic protein into prion. In this respect it is envisioned that the reaction mixture could further comprise a test compound. Control reaction mixtures and reaction mixtures including the test compound could be accessed for levels of prion following amplification. Wherein a difference between the levels of prion in the test versus control reaction mixtures is detected, compounds could be identified that either enhance or inhibit conversion of non-pathogenic protein to prion. In further embodiments of this method samples from control and test reaction mixtures may be taken after two, three, four or more amplification steps to determine a rate of prion replication. By comparing the rate of control prion replication versus the rate of propagation in the presence of a test compound candidate modifiers could be quantitatively accessed for their effect on prion replication.

I. Transmissible Spongiform Encephalopathies (TSES)

In animals the most common TSE is scrapie, but the most famous and dangerous disease is the recently discovered BSE, which affects cattle and is known in the world over by its lay term “mad cow disease.” In humans the most common TSE is CJD, which occurs worldwide with an incidence of 0.5 to 1.5 new cases per one million people each year (Johnson and Gibbs, Jr., 1998). Three different forms of CJD have been traditionally recognized (Collinge, 2001): sporadic (sCJD; 85% of cases), familial (fCJD; 10%), and iatrogenic (iCJD; ˜5%). However, in 1996, a new variant form of CJD (vCJD) emerged in the UK (Will et al., 1996), which has been associated with consumption of meat infected with BSE (Bruce, 2000; Collinge, 1999; Scott et al., 1999). In contrast with typical cases of sCJD, vCJD affects young patients with an average age of 27 years old, and is a relatively long illness (14 months compared with 4.5 months for sCJD). Because of insufficient information available about the incubation time and the levels of exposure to contaminated cattle food products it is impossible to make well-founded predictions about the potential future incidence of vCJD (Balter, 2001). In animals there is no evidence for inherited forms of the disease and most cases appear to be acquired by horizontal or vertical transmission.

The clinical diagnosis of sCJD is based on a combination of rapidly progressive multifocal dementia with pyramidal and extrapyramidal signs, myoclonus, and visual or cerebellar signs, associated with a characteristic periodic electroencephalogram (EEG) (Collins et al., 2000; Ingrosso et al., 2002; Kordek, 2000; Weber et al., 1997). A key feature for diagnosing sCJD, and distinguishing it from Alzheimer's disease and other dementias, is the rapid progression of clinical symptoms and the short duration of the disease, which is often less than 2 years. The clinical manifestation of fCJD is very similar, except that the disease onset is slightly earlier than in sCJD. Family history of inherited CJD or genetic screening for mutations in the PrP gene are used to establish fCJD diagnosis, although lack of family history does not excludes an inherited origin (Kordek, 2000).

Variant CJD appears initially as a progressive neuropsychiatric disorder characterized by symptoms of anxiety, depression, apathy, withdrawal and delusions (Henry and Knight, 2002). This is combined with persistent painful sensory symptoms and is followed by ataxia, myoclonus, and dementia. Variant CJD is differentiated from sCJD by the duration of illness (usually longer than 6 months) and EEG analysis (vCJD does not show the atypical pattern observed in sCJD). A high bilateral pulvinar signal noted during MRI is usually used to help diagnose vCJD (Coulthard et al., 1999). In addition, a tonsil biopsy may be used to help diagnose vCJD, based on a number of cases of vCJD have been shown to test positive for PrP^{Sc} staining in lymphoid tissue (such as tonsil and appendix). However, because of the invasive nature of this test, it should be performed only in patients who fulfill the clinical criteria of vCJD where the MRI of the brain does not show the characteristic pulvinar sign (Hill et al., 1999).

GSS is a dominantly inherited illness that is characterized by dementia, Parkinsonian symptoms, and a relatively long duration (typically, 5-8 years) (Boellaard et al., 1999; Ghetti et al., 1995). Clinically, GSS is similar to Alzheimer's disease, except that is often accompanied by ataxia and seizures. Diagnosis is established by clinical examination and genetic screening for PrP mutations (Ghetti et al., 1995). FFI is also dominantly inherited and associated to PrP mutations. However, the major clinical finding associated with FFI is insomnia, followed at late stages by myoclonus, hallucinations, ataxia, and dementia (Cortelli et al., 1999).

II. Protein Sources

A. Sources of Non-Pathogenic Protein

As detailed above, a variety of sources may be used to obtain non-pathogenic protein for use in the methods of the invention. For instance the protein maybe endogenously expressed in cells and these cells used to make a lysate that provides the non-pathogenic protein. The lysate may be from tissue culture cells, or extracted from whole organisms, organs, or tissues. For example, in the case where the non-pathogenic protein is PrP brain homogenates may be used. These brain homogenates may be mammalian brain homogenates, and it may be preferable that they be from the same species as the particular sample being tested or from transgenic mice engineered to express PrP from the specie to be tested.

It is envisioned that in addition to using crude cell lysates partially purified protein may also be used. For instance in the case of PrP it has been shown that the majority of the protein localizes to the membrane in structures known as “lipid-rafts.” Thus partial purification of PrP^{C} can be achieved by enriching the lysate for lipid-rafts. Methods for this enrichment typically rely on the resistance of lipid-raft structures to mild detergent, such as ice-cold Triton X-100, and are well known to those in the art.

As indicated above it may in some cases be preferable that the non-pathogenic protein be deglycosylated. For example non-pathogenic protein may be treated with peptide N-glycosidase F (New England Biolabs, Beverly, Mass.) according to the manufacturers instructions. In this case, incubation for about 2 h at 37° C. results in significant deglycosylation.

Generally, “purified” will refer to a non-pathogenic protein composition that has been subjected to fractionation or isolation to remove various other protein or peptide components, and which composition substantially retains non-pathogenic protein, as may be assessed, for example, by Western blot to detect the non-pathogenic protein.

To purify non-pathogenic protein from natural or recombinant composition the composition will be subjected to fractionation to remove various other components from the composition. Various techniques suitable for use in protein purification will be well known to those of skill in the art. These include, for example, precipitation with ammonium sulfate, PTA, PEG, antibodies and the like, or by heat denaturation followed by centrifugation; chromatography steps such as ion exchange, gel filtration, reverse phase, hydroxylapatite, lectin affinity and other affinity chromatography steps; isoelectric focusing; gel electrophoresis; and combinations of such and other techniques.

In some embodiments of the invention the source of the non-pathogenic protein maybe from cells made or engineered to over express the protein. For instance cells may be transformed with a nucleic acid vector that expresses the non-pathogenic protein, for example PrP^{C}. These cells may comprise mammalian cells, bacterial cells, yeast cell, insect cells, whole organisms, such as transgenic mice, or other cells that may be a useful source of the non-pathogenic protein. Raw cell lysates or purified non-pathogenic protein from expressing cells may be used as the source of the non-pathogenic protein.

In some cases it may be preferable that the recombinant protein be fused with additional amino acid sequence. For example over expressed protein may be tagged for purification or to facilitate detection of the protein in a sample. Some possible fusion proteins that could be generated include histadine tags, Glutathione S-transferase (GST), Maltose binding protein (MBP)), green fluorescent protein (GFP), Flag and myc tagged PrP. These additional sequences may be used to aid in purification and/or detection of the recombinant protein, and in some cases may then be removed by protease cleavage. For example coding sequence for a specific protease cleavage site may be inserted between the non-pathogenic protein coding sequence and the purification tag coding sequence. One example for such a sequence is the cleavage site for thrombin. Thus fusion proteins may be cleaved with the protease to free the non-pathogenic protein from the purification tag.

In the case where non-pathogenic protein is highly purified the reaction mix may further comprise additional cell lysate to provide secondary factors important for conversion. For example in the case of PrP^{C}, brain homogenate from PrP null mice may be ideal. It is contemplated that the method of the invention might be used to identify co-factors important in pathogenic conversion of non-pathogenic protein.

Any of the wide variety of vectors known to those of skill in the art could be used to over express non-pathogenic protein. For example, plasmids or viral vectors may be used. It is well understood to these of skill in the art that these vectors may be introduced into cells by a variety of methods including, but not limited to, transfection (e.g, by liposome, calcium phosphate, electroporation, particle bombardment, etc.), transformation, and viral transduction.

Non-pathogenic protein may further comprise proteins that have amino sequence containing substitutions, insertions, deletions, and stop codons as compared to wild type sequence. In certain embodiments of the invention, a protease cleavage sequence may be added to allow inactivation of protein after it is converted into prion form. For example cleavage sequences recognized by Thrombin, Tobacco Etch Virus (Life Technologies, Gaithersburg, Md.) or Factor Xa (New England Biolabs, Beverley, Mass.) proteases may be inserted into the sequence.

In certain embodiments changes may be made in the PrP coding sequence for example in the coding sequence for mouse, human, bovine, sheep, goat, and/or elk PrP, as give by GenBank accession numbers NM_{—}011170, NM_{—}183079, AY335912, AY723289, AY723292 and AY748455 respectively, all of which are incorporated herein by reference. For example mutations could be made to match a variety of mutations and polymorphisms known for various mammalian PrP genes (Table 2). It is contemplated that cells expressing these altered PrP genes may be used as a source of the non-pathogenic protein. These cells may comprise cells that endogenously express the mutant PrP gene or cells that have been made to express a mutant PrP protein by the introduction of an expression vector. Use of a mutated non-pathogenic protein may be of particular advantage, as it is possible that these proteins may be more easily converted to prion, and thus may further enhance the sensitivity of the method of the invention.

It is contemplated that the method of the current invention may be used to test the effect of mutations on the conversion rates of non-pathogenic proteins. For example in case of PrP, mutant PrP, and wild type PrP be mixed with equal amounts of prion and saPMCA performed. By comparing the rate of prion replication in samples with mutant PrP versus wild type PrP mutations could be identified that modulate the ability of prion to replicate. Further results from such studies could be used to determine whether animals with certain PrP polymorphisms are more or less susceptible to TSEs.

TABLE 2

| Pathogenic human | Human | Sheep | Bovine |
|---|---|---|---|
| mutations | polymorphisms | polymorphisms | polymorphisms |
| 2 octarepeat insert | Codon 129 | Codon 171 | 5 or 6 octarepeats |
| | Met/Val | Arg/Glu | |
| 4-9 octarepeat insert | Codon 219 | Codon 136 | |
| | Glu/Lys | Ala/Val | |
| Codon 102 Pro-Leu | | | |
| Codon 105 Pro-Leu | | | |
| Codon 117 Ala-Val | | | |
| Codon 145 Stop | | | |
| Codon 178 Asp-Asn | | | |
| Codon 180 Val-Ile | | | |
| Codon 198 Phe-Ser | | | |
| Codon 200 Glu-Lys | | | |
| Codon 210 Val-Ile | | | |
| Codon 217 Asn-Arg | | | |
| Codon 232 Met-Ala | | | |

B. Sources of Samples for saPMCA Assay

As described above it is contemplated that samples used in the methods of the invention may essentially comprise any composition capable of being contaminated with a prion. Such compositions could comprise tissue samples from tissues including, but not limited to, blood, lymph nodes, brain, spinal cord, tonsils, spleen, skin, muscles, appendix, olfactory epithelium, cerebrospinal fluid, urine, milk, intestines, tears and/or saliva. Other compositions from which samples may be taken for analysis comprise food stuffs, drinking water, forensic evidence, surgical implements, and/or machinery.

C. Methods For Detecting Prion in saPMCA Reaction Mixes

Direct and indirect methods may be used for detection of prion protein in a reaction mix or serial reaction mix. For methods in which prion is directly detected separation of newly formed prion from remaining non-pathogenic protein is usually required. This is typically accomplished based on the different nature of prion versus non-pathogenic protein for instance prion is typically highly insoluble and resistant to protease treatment. Therefore in the case or PrP^{Sc} and PrP^{C} separation can be by either protease treatment, or differential centrifugation in a detergent, or a combination of the two techniques.

In the case where prion and non-pathogenic protein are separated by protease treatment, reaction mixtures are incubated with, for example, Proteinase K (PK). An exemplary proteinase treatment comprises digestion of the protein, e.g., PrP^{C}, in the reaction mixture with 50 μg/ml of proteinase K (PK) for about 1 hour at 45° C. Reactions with PK may be stopped prior to assessment of prion levels by addition of PMSF or electrophoresis sample buffer. Incubation at 45° C. with 50 μg/ml of PK is sufficient to remove non-pathogenic protein.

In some cases non-pathogenic protein may be separated from prion by fractionation. In the case of PrP^{C} and PrP^{Sc} differential solubility may be used. An exemplary procedure comprises; incubating the reaction mixture in the presence of 10% sarkosyl for 30 min at 4° C. Thereafter, samples are centrifuged at 100,000 x g for 1 hr in a Biosafe Optima MAX ultracentrifuge (Beckman Coulter, Fullerton, Calif.) and the pellet, which contains the PrP^{Sc}, is resuspended then analyzed for prion. In some cases prior to the addition of sarkosyl, reaction mixtures are incubated with different concentrations of guanidine hydrochloride for 2 hr at room temperature with shaking. Thereafter, sarkosyl is added and the soluble and insoluble proteins are separated using centrifugation.

Prion might also be separated from the non-pathogenic protein by the use of ligands that specifically bind and precipitated the misfolded form of the protein, including conformational antibodies, certain nucleic acids, plasminogen, PTA and/or various peptide fragments (Soto et al., 2004).

1. Western Blot

Reaction mixtures fractioned or treated with protease to remove PrP^{C} may be subjected to Western blot for detection of PrP^{Sc}. Typical Western blot procedures begin with fractionating proteins by sodium dodecyl sulphate-polyacrylamide gel electrophoresis (SDS-PAGE) under reducing conditions. The proteins are then electroblotted onto a membrane, such as nitrocellulose or PVDF and probed, under conditions effective to allow immune complex (antigen/antibody) formation, with an anti-prion antibody. An exemplary antibody for detect of PrP is the 3F4 monoclonal antibody (Kascsak et al., 1987). Following complex formation the membrane is washed to remove non-complexed material. A preferred washing procedure includes washing with a solution such as PBS/Tween, or borate buffer. The immunoreactive bands are visualized by a variety of assays known to those in the art. For example the enhanced chemoluminesence assay (ECL) (Amersham, Piscataway, N.J.).

Prion concentration may be estimated by Western blot followed by densitometric analysis, and comparison to Western blots of samples for which the concentration of prion is known. For example this may be accomplished by scanning data into a computer followed by analysis with quantiation software. To obtain a reliable and robust quantification, several different dilutions of the sample are analyzed in the same gel.

2. ELISA and Confromation Dependent Immunoassay (CDI)

As detailed above, immunoassays in their most simple and direct sense are binding assays. Certain preferred immunoassays are the various types of enzyme linked immunosorbent assays (ELISAs), radioimmunoassays (RIA), and specifically conformation-dependent immunoassays (CDI) known in the art.

In one exemplary ELISA, the anti-prion antibodies are immobilized onto a selected surface exhibiting protein affinity, such as a well in a polystyrene microtiter plate. Then, reaction mixture suspected of containing prion protein antigen, is added to the wells. After binding and washing to remove non-specifically bound immune complexes, the bound prion protein may be detected. Detection is generally achieved by the addition of another anti-prion antibody that is linked to a detectable label. This type of ELISA is a simple “sandwich ELISA.” Detection may also be achieved by the addition of a second anti-prion antibody, followed by the addition of a third antibody that has binding affinity for the second antibody, with the third antibody being linked to a detectable label.

In another exemplary ELISA, the reaction mixture suspected of containing the prion protein antigen are immobilized onto the well surface and then contacted with the anti-prion antibodies. After binding and washing to remove non-specifically bound immune complexes, the bound anti-prion antibodies are detected. Where the initial anti-prion antibodies are linked to a detectable label, the immune complexes may be detected directly. Again, the immune complexes may be detected using a second antibody that has binding affinity for the first anti-prion antibody, with the second antibody being linked to a detectable label.

Another ELISA in which protein of the reaction mix is immobilized, involves the use of antibody competition in the detection. In this ELISA, labeled antibodies against prion protein are added to the wells, allowed to bind, and detected by means of their label. The amount of prion protein antigen in a given reaction mix is then determined by mixing it with the labeled antibodies against prion before or during incubation with coated wells. The presence of prion protein in the sample acts to reduce the amount of antibody against prion available for binding to the well and thus reduces the ultimate signal. Thus the amount of prion in the sample may be quantified.

Irrespective of the format employed, ELISAs have certain features in common, such as coating, incubating or binding, washing to remove non-specifically bound species, and detecting the bound immune complexes. These are described below.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein and solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the biological sample to be tested under conditions effective to allow immune complex (antigen/antibody) formation. Detection of the immune complex then requires a labeled secondary binding ligand or antibody, or a secondary binding ligand or antibody in conjunction with a labeled tertiary antibody or third binding ligand.

“Under conditions effective to allow immune complex (antigen/antibody) formation” means that the conditions preferably include diluting the antigens and antibodies with solutions such as BSA, bovine gamma globulin (BGG) and phosphate buffered saline (PBS)/Tween. These added agents also tend to assist in the reduction of nonspecific background.

The “suitable” conditions also mean that the incubation is at a temperature and for a period of time sufficient to allow effective binding. Incubation steps are typically from about 1 to 2 to 4 hours, at temperatures preferably on the order of 25° C. to 27° C., or may be overnight at about 4° C. or so.

Following all incubation steps in an ELISA, the contacted surface is washed so as to remove non-complexed material. A preferred washing procedure includes washing with a solution such as PBS/Tween, or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. Preferably, this will be an enzyme that will generate color development upon incubating with an appropriate chromogenic substrate. Thus, for example, one will desire to contact and incubate the first or second immune complex with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea and bromocresol purple or 2,2′-azino-di-(3-ethyl-benzthiazoline-6-sulfonic acid [ABTS] and H₂O₂, in the case of peroxidase as the enzyme label. Quantification is then achieved by measuring the degree of color generation, e.g., using a visible spectra spectrophotometer.

3. Animal Bioassy

The presence of prion in reaction mixtures may additionally be detected indirectly by the animal bioassay that is well known to those of skill in the art. In the case of PrP^{Sc} an exemplar procedure may comprise:

Animals (Syrian Golden hamsters) 4- to 6-weeks old are anesthetized and injected stereotaxically in the right hippocampus with about 1 μl of the reaction mix. This may be accomplished using a computerized perfusion machine that delivers the sample into the brain at a given rate, for example 0.1 μl/min. The onset of clinical disease is measured by scoring the animals twice a week using the following scale:
1. Normal animal;
2. Mild behavioral abnormalities including hyperactivity and hypersensitivity to noise;
3. Moderate behavioral problems including tremor of the head, ataxia, wobbling gait, head bobbing, irritability and aggressiveness;
4. Severe behavioral abnormalities including all of the above plus jerks of the head and body and spontaneous backrolls;
5. Terminal stage of the disease in which the animal lies in the cage and is no longer able to stand up.

Animals scoring level 4 during two consecutive weeks are considered sick and are sacrificed. Sacrifice may be by exposition to carbon dioxide to avoid excessive pain. Brains and other tissues are extracted and analyzed histologically by methods that are well known in the art. For instance one hemisphere is fixed in 10% formaldehyde solution, cut in sections and embedded in paraffin. Serial sections (˜6 μm thick) from each block are stained with hematoxylin-eosin, using standard protocols or incubated with antibodies recognizing PrP, in some cases incubation with an antibody to the glial fibrillary acidic protein may be used as a control. Immunoreactions are developed, for example using the peroxidase-antiperoxidase methods. In this case antibody specificity is verified by absorption. In some cases biochemical examination for PrP^{Sc} using Western blot analysis may also be used. In some case both histologic and biochemical analyses may be undertaken, by using one brain hemisphere for each.

4. Cellular Assays

Another strategy to detect low concentrations of prions is the use of cell infectivity assays (Klohn et al., 2003). Mouse neuroblastoma N2a sublines are highly susceptible to certain prions, as evidenced by accumulation of PrP^{res} and infectivity. In this assay, susceptible N2a cells are exposed to prion-containing samples for 3 days, grown to confluence, and split three times. The proportion of PrP^{res}-containing cells is determined with automated counting equipment. In certain applications the number of prion containing cells may also be determined by flow cytometry. The dose-response to infection is linear over two logs of prion concentrations. The cell assay was claimed to be as sensitive as the mouse bioassay, 10 times faster, 2 orders of magnitude less expensive, and suitable for automation by use of robots.

D. PrP^{C} Labeling:

In certain applications of the present invention, the non-pathogenic protein can be labeled to enable high sensitivity of detection of protein that is converted into prion. For example, non-pathogenic protein may be radioactively labeled, epitope tagged, or fluorescently labeled. The label may be detected directly or indirectly. Radioactive labels include, but are not limited to ¹²⁵I, ³²P, ³³P, and ³⁵S.

The mixture containing the labeled protein is subjected to saPMCA and the product detected with high sensitivity by following conversion of the labeled protein after removal of the unconverted protein for example by proteolysis. Alternatively, the protein could be labeled in such a way that a signal can be detected upon the conformational changes induced during conversion. An example of this is the use of FRET technology, in which the protein is labeled by two appropriate fluorophers, which upon refolding become close enough to exchange fluorescence energy (see for example U.S. Pat. No. 6,855,503).

1. Fluorescence Resonance Energy Transfer (FRET) One class of dyes which have been developed to give large and different Stokes shifts, based on the Fluorescence Resonance Energy Transfer (FRET) mechanism and used in the simultaneous detection of differently labeled samples in a mixture, are the ET (Energy Transfer) dyes. These ET dyes include a complex molecular structure consisting of a donor fluorophore and an acceptor fluorophore as well as a labeling function to allow their conjugation to biomolecules of interests. Upon excitation of the donor fluorophore, the energy absorbed by the donor is transferred by the FRET mechanism to the acceptor fluorophore and causes it to fluoresce. Different acceptors can be used with a single donor to form a set of ET dyes so that when the set is excited at one single donor frequency, various emissions can be observed depending on the choice of the acceptors. Upon quantification of these different emissions, changes in the folding of a labeled protein may be rapidly determined. Some exemplary dyes that may be used comprise BODIPY FL, fluorescein, tetmethylrhodamine, IAEDANS, EDANS or DABCYL. Other dyes have also been used for FRET for examples dyes disclosed in U.S. Pat. Nos. 5,688,648, 6,150,107, 6,008,373 and 5,863,727 and in PCT publications WO 00/13026, and WO 01/19841, all incorporated herein by reference.

III. Antibody Generation

In certain embodiments, the present invention involves antibodies. For example, antibodies are used in many of the method for detecting prion (e.g. Western blot and ELISA). In addition to antibodies generated against full length proteins, antibodies also may be generated in response to smaller constructs comprising epitopic core regions, including wild-type and mutant epitopes.

As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting.

Monoclonal antibodies (mAbs) are recognized to have certain advantages, e.g., reproducibility and large-scale production, and their use is generally preferred. The invention thus provides monoclonal antibodies of the human, murine, monkey, rat, hamster, rabbit and even chicken origin.

The term “antibody” is used to refer to any antibody-like molecule that has an antigen binding region, and includes antibody fragments such as Fab′, Fab, F(ab′)₂, single domain antibodies (DABs), Fv, scFv (single chain Fv), and the like. The techniques for preparing and using various antibody-based constructs and fragments are well known in the art. Means for preparing and characterizing antibodies are also well known in the art (See, e.g., Harlow and Lane, “Antibodies: A Laboratory Manual,” Cold Spring Harbor Laboratory, 1988; incorporated herein by reference).

The methods for generating monoclonal antibodies (mAbs) generally begin along the same lines as those for preparing polyclonal antibodies. Briefly, a polyclonal antibody may be prepared by immunizing an animal with an immunogenic polypeptide composition in accordance with the present invention and collecting antisera from that immunized animal. Alternatively, in some embodiments of the present invention, serum is collected from persons who may have been exposed to a particular antigen. Exposure to a particular antigen may occur in a work environment, such that those persons have been occupationally exposed to a particular antigen and have developed polyclonal antibodies to a peptide, polypeptide, or protein. In some embodiments of the invention polyclonal serum from occupationally exposed persons is used to identify antigenic regions in a prion through the use of immunodetection methods.

A wide range of animal species can be used for the production of antisera. Typically the animal used for production of antisera is a rabbit, a mouse, a rat, a hamster, a guinea pig or a goat. Because of the relatively large blood volume of rabbits, a rabbit is a preferred choice for production of polyclonal antibodies.

As is well known in the art, a given composition may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin also can be used as carriers. Means for conjugating a polypeptide to a carrier protein are well known in the art and include glutaraldehyde, m-maleimidobenzoyl-N-hydroxysuccinimide ester, carbodiimide and bis-biazotized benzidine.

As also well known in the art, the immunogenicity of a particular immunogen composition can be enhanced by the use of non-specific stimulators of the immune response, known as adjuvants. Suitable moleculer adjuvants include all acceptable immunostimulatory compounds, such as cytokines, toxins or synthetic compositions.

Adjuvants that may be used include IL-1, IL-2, IL-4, IL-7, IL-12, γ-interferon, GMCSP, BCG, aluminum hydroxide, MDP compounds, such as thur-MDP and nor-MDP, CGP (MTP-PE), lipid A, and monophosphoryl lipid A (MPL). RIBI, which contains three components extracted from bacteria, MPL, trehalose dimycolate (TDM) and cell wall skeleton (CWS) in a 2% squalene/Tween 80 emulsion also is contemplated. MHC antigens may even be used. Exemplary, often preferred adjuvants include complete Freund's adjuvant (a non-specific stimulator of the immune response containing killed Mycobacterium tuberculosis), incomplete Freund's adjuvants and aluminum hydroxide adjuvant.

In addition to adjuvants, it may be desirable to co-administer biologic response modifiers (BRM), which have been shown to upregulate T cell immunity or downregulate suppressor cell activity. Such BRMs include, but are not limited to, Cimetidine (CIM; 1200 mg/d) (Smith/Kline, PA); low-dose Cyclophosphamide (CYP; 300 mg/m²) (Johnson/ Mead, NJ), cytokines such as γ-interferon, IL-2, or IL-12 or genes encoding proteins involved in immune helper functions, such as B-7.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization.

A second, booster injection also may be given. The process of boosting and titering is repeated until a suitable titer is achieved. When a desired level of immunogenicity is obtained, the immunized animal can be bled and the serum isolated and stored, and/or the animal can be used to generate mAbs.

mAbs may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified polypeptide, peptide or domain, be it a wild-type or mutant composition. The immunizing composition is administered in a manner effective to stimulate antibody producing cells.

mAbs may be further purified, if desired, using filtration, centrifugation and various chromatographic methods such as HPLC or affinity chromatography. Fragments of the monoclonal antibodies of the invention can be obtained from the monoclonal antibodies so produced by methods which include digestion with enzymes, such as pepsin or papain, and/or by cleavage of disulfide bonds by chemical reduction. Alternatively, monoclonal antibody fragments encompassed by the present invention can be synthesized using an automated peptide synthesizer.

It also is contemplated that a molecular cloning approach may be used to generate mAbs. For this, combinatorial immunoglobulin phagemid libraries are prepared from RNA isolated from the spleen of the immunized animal, and phagemids expressing appropriate antibodies are selected by panning using cells expressing the antigen and control cells. The advantages of this approach over conventional hybridoma techniques are that approximately 104 times as many antibodies can be produced and screened in a single round, and that new specificities are generated by H and L chain combination which further increases the chance of finding appropriate antibodies.

IV. Screening for Modulators of the Prion Function

As described above the current invention may be used to identify compounds that modify the ability of prions to replicate, such compounds would be candidates for treatment of prion mediated disease. It is envisioned that the method for screening compounds could comprise performing saPMCA on control reaction mixtures and reaction mixtures including the test compound could be accessed for levels of prion following amplification. Wherein a difference between the levels of prion in the test versus control reaction mixtures is detected, compounds could be identified that either enhance or inhibit conversion of non-pathogenic protein to prion. These assays may comprise random screening of large libraries of candidate substances; alternatively, the assays may be used to focus on particular classes of compounds selected with an eye towards structural attributes that are believed to make them more likely to modulate the function of prions.

By function, it is meant that one may determine the efficiency of conversion by assaying conversion of a standard amount of non-pathogenic protein into prion by a known amount of prion. This may be determined by, for instance, quantitating the amount of prion in a reaction mix following a certain number of cycles of saPMCA. This is shown more specifically in Example 2 below wherein both an enhancer of prion replication and an inhibitor of prion replication are identified. Specifically it is shown that addition of Cu²⁺ to the reaction mixture inhibits prion replication, while addition of EDTA to the reaction mix enhances prion conversion. Due to the rapid, high throughput nature of the saPMCA assay disclosed herein it is envisioned that panels of potential prion replication modulators may be screened.

It will, of course, be understood that all the screening methods of the present invention are useful in themselves notwithstanding the fact that effective candidates may not be found or identified. The invention provides methods for screening for such candidates, not solely methods of finding them.

A. Modulators

As used herein the term “candidate substance” refers to any molecule that may potentially inhibit or enhance prion function activity. The candidate substance may be a protein or fragment thereof, a small molecule, or even a nucleic acid molecule. It may prove to be the case that the most useful pharmacological compounds will be compounds that are structurally related to PrP, or other copper binding molecules. Using lead compounds to help develop improved compounds is know as “rational drug design” and includes not only comparisons with know inhibitors and activators, but predictions relating to the structure of target molecules.

The goal of rational drug design is to produce structural analogs of biologically active polypeptides or target compounds. By creating such analogs, it is possible to fashion drugs, which are more active or stable than the natural molecules, which have different susceptibility to alteration or which may affect the function of various other molecules. In one approach, one would generate a three-dimensional structure for a target molecule, or a fragment thereof. This could be accomplished by x-ray crystallography, computer modeling or by a combination of both approaches.

It also is possible to use antibodies to ascertain the structure of a target compound activator or inhibitor. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

On the other hand, one may simply acquire, from various commercial sources, small molecule libraries that are believed to meet the basic criteria for useful drugs in an effort to “brute force” the identification of useful compounds. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds modeled on active, but otherwise undesirable compounds.

Candidate compounds may include fragments or parts of naturally-occurring compounds, or may be found as active combinations of known compounds, which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be peptide, polypeptide, polynucleotide, small molecule inhibitors or any other compound(s) that may be designed through rational drug design starting from known inhibitors or stimulators.

Other suitable modulators include antibodies (including single chain antibodies), each of which would be specific for the target molecule. Such compounds are described in greater detail elsewhere in this document.

In addition to the modulating compounds initially identified, the inventors also contemplate that other sterically similar compounds may be formulated to mimic the key portions of the structure of the modulators. Such compounds, which may include peptidomimetics of peptide modulators, may be used in the same manner as the initial modulators. Preferred modulators of prion replication would have the ability to cross the blood-brain barrier since a large number of prion manifest themselves in the central nervous system.

An inhibitor according to the present invention may be one which exerts its activity directly on the prion, on the non-pathogenic protein or on factors required for the conversion of non-pathogenic protein to prion. Regardless of the type of inhibitor or activator identified by the present screening methods, the effect of the inhibition or activation by such a compound results in altered prion amplification or replication as compared to that observed in the absence of the added candidate substance.

V. Kits

Any of the compositions described herein may be comprised in a kit. In a non-limiting example, non-pathogenic protein, prion conversion factors, decontamination solution and/or conversion buffer with a metal chelator are provided in a kit. The kit may further comprise reagents for expressing or purifying non-pathogenic protein. The kit may also comprise reagents that may be used to label the non-pathogenic protein, with for example, radio isotopes or fluorophors.

Kits for implementing methods of the invention described herein are specifically contemplated. In some embodiments, there are kits for amplification and detection of prion in a sample. In these embodiments, a kit can comprise, in suitable container means, 1, 2, 3, 4, 5, 6, 7, 8, 9, 10, 11, 12 or more of the following: 1) a conversion buffer; 2) non-pathogenic protein; 3) decontamination solution; 4) a positive control, prion containing sample; 5) a negative control sample, not containing prion; or 6) reagents for detection of prion.

Regents for the detection of prion can comprise one or more of the following: pre coated microtiter plates for ELISA and/or CDI detection of prion; tissue culture cells in which prion can replicate; or antibodies for use in ELSA, CDI or Western blot detection methods.

Additionally, kits of the invention may contain one or more of the following: protease free water; copper salts for inhibiting prion replication; EDTA solutions for enhancing prion replication; Proteinase K for the separation of prion from non-pathogenic protein; fractionation buffers for the separation of prion from non-pathogenic, modified, or labeled proteins (increase sensitivity of detection); or conversion factors (enhance efficiency of amplification).

In certain embodiments the conversion buffer may be supplied in a “ready for amplification format” where it is allocated in a microtiter plate such that the sample and non-pathogenic protein may be added to first well, and subjected to primary amplification. There after a portion of the reaction mix is moved to an adjacent well and additional non-pathogenic protein added for serial amplification. These steps many be repeated across the microtiter plate for multiple serial amplifications.

The components of the kits may be packaged either in aqueous media or in lyophilized form. The container means of the kits will generally include at least one vial, test tube, plate, flask, bottle, syringe or other container means, into which a component may be placed, and preferably, suitably aliquoted. Where there is more than one component in the kit (labeling reagent and label may be packaged together), the kit also will generally contain a second, third or other additional container into which the additional components may be separately placed. However, various combinations of components may be comprised in a vial. The kits of the present invention also will typically include a means for containing proteins, and any other reagent containers in close confinement for commercial sale. Such containers may include injection or blow-molded plastic containers into which the desired vials are retained.

When components of the kit are provided in one and/or more liquid solutions, the liquid solution is typically an aqueous solution that is sterile and proteinase free. In some cases proteinatious compositions may be lyophilized to prevent degradation and/or the kit or components thereof may be stored at a low temperature (i.e. less than about 4° C.). When reagents and/or components are provided as a dry powder and/or tablets, the powder can be reconstituted by the addition of a suitable solvent. It is envisioned that the solvent may also be provided in another container means.

### EXAMPLES

The following examples are included to further illustrate various aspects of the invention. It should be appreciated by those of skill in the art that the techniques disclosed in the examples that follow represent techniques and/or compositions discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Purification of PrP^{Sc} from Brain

One gram of brain tissue was homogenized in 5 ml of cold PBS containing protease inhibitors. For PMCA-generated PrP^{res}, after the last amplification, the total sample containing the normal brain homogenate used as a substrate was processed in the same way as brain homogenate. The samples were mixed with 1 volume of 20% sarkosyl and the mixture was homogenized and sonicated until a clear preparation was obtained. Samples were centrifuged at 5000 rpm for 15 min at 4° C. The pellet was discarded and the supernatant was mixed with ⅓ volume of PBS containing 0.1% SB-314 and samples were centrifuged in a Biosafe Optima MAX ultracentrifuge (Beckman Coulter, Fullerton, Calif.) at 100,000×g for 3 hr at 4° C. Supernatant was discarded and pellets were resuspended in 600 μl of PBS containing 0.1% SB-314, 10% NaCl and sonicated. The resuspended pellet was layered over 600 ul of PBS containing 20% saccharose, 10% NaCl and 0.1% SB 3-14 and centrifuged for 3 h at 4 ° C. The supernatant was discarded and the pellet resuspended in 300 μl of PBS containing 0.1% SB 3-14 and sonicated again. After sonication the samples were incubated with PK (100 μg/ml) for 2 hr at 37° C. and shaking. The digested sample was layered over 100 μl of PBS containing 20% Sarkosyl, 0.1% SB 3-14 and 10% NaCl and centrifuged for 1 h 30 min at 100,000×g. The final pellet was resuspended in 100 μl of PBS and sonicated. The sample was stored at −80 ° C. Purity was analyzed by silver staining and amino acid composition analysis.

### Example 2

### Automation: Increase throughput and decrease on Time for Amplification

The use of a single-probe traditional sonicator imposes a practical problem for handling many samples simultaneously, as a diagnostic test would require. The inventors have adapted the cyclic amplification system to a 96-well format microplate sonicator (the Misonix™ Model 3000 (Farmingdale, N.Y.)), which provides sonication to all the wells at the same time and can be programmed for automatic operation. This improvement not only decreases processing time, increase throughput, and allows performing routinely many more cycles than single-probe sonicator, but also prevents loss of material. Cross contamination is eliminated since there is no direct probe intrusion into the sample. The latter is essential to handle infectious samples and to minimize false positive results. Ten cycles of 1 h incubation followed by sonication pulses of 30 seconds gave a significant amplification of PrP^{Sc} signal, similar to that observed using a traditional sonicator.

For practical applications in routine diagnosis, it is desirable to reduce the time it takes to perform the test. In order to evaluate whether the time can be reduced the inventors studied the efficiency of amplification using various incubation times between sonications. The efficiency of amplification in automated PMCA was best when the samples were incubated for 30 min. Therefore, in all the following studies each cycle consisted of a 30 minute incubation followed by a pulse of sonication. This improvement let to a 50% shortening in time compared to the traditional PMCA procedure.

### Example 3

### Increase on Amplification Efficiency by Metal Chelation

As part of our efforts to optimize the PMCA procedure the inventors discovered that the relatively low level of amplification observed previously was in part due to the presence of metal cations in the samples. When the PMCA reactions were done in the presence of EDTA, a broad-range metal chelator, the efficiency of amplification was dramatically higher.

It is well established that PrP binds copper (and to a lesser extent zinc) with high affinity and indeed a possible biological function for the normal prion protein is to participate in Cu²⁺ transport across the cell membrane (Brown and Sassoon, 2002). The results indicate that the positive effect of EDTA in boosting PMCA efficiency was lost when Cu²⁺ was added to the reaction. The effect is very clear and is concentration-dependent. No significant effect was observed with other divalent cations such as Ca²⁺ and Mg²⁺, but Zn²⁺ also decreased efficiency of prion conversion, although less marked than Cu²⁺. These findings suggest that binding of Cu²⁺ (or Zn²⁺) to PrPC may inhibit the prion replication process, suggesting a possible novel therapeutic approach for prion diseases. This data helped to dramatically increase PMCA efficiency by adding a metal chelator (such as EDTA) to the reaction.

### Example 4

### Ultrasensitive Detection of Prions by saPMCA

Sensitivity of detection after automated PMCA was analyzed by comparing the signal intensity in Western blots before and after amplification. It was determined that 140 PMCA cycles enabled detection of PrP^{Sc} in as little as a 6.6 million-fold dilution of 263K scrapie brain. An equivalent quantity of PrP^{Sc} was detected without PMCA in a 1,000-fold dilution of the same material, indicating that the increase of sensitivity under these conditions was approximately 6,600-fold.

During these studies, it was noted that the efficiency of amplification started to decrease after around 150 cycles (75 h of incubation). The inventors discovered that the reason for this problem is the inactivation of the material by continuous incubation at 37° C. It is likely that incubation may have a negative effect on the stability of PrP^{C} substrate or other brain cofactors essential to catalyze the conversion. This conclusion was based on an experiment in which the amplification efficiency was dramatically reduced when the 10% normal brain homogenate was pre-incubated (with our without sonication) during 72 h prior to the beginning of PMCA amplifications. This result was motivation to develop a new technology called serial automated PMCA (saPMCA) in order to further increase sensitivity of detection. This technology consists of performing a series of up to 144 PMCA cycles each. After the end of a first round of 144 PMCA cycles, samples are diluted 10-fold into fresh 10% normal brain homogenate and another 144 (or less) PMCA cycles is performed. saPMCA resolves the problem of exhaustion of the substrate and enables to maintain the exponential conversion of PrP. Two successive rounds of PMCA cycling separated by a 10-fold dilution of the amplified samples into fresh 10% normal brain homogenate, led to a dramatic increase in sensitivity. The experiment consisted of performing a first round of 96 PMCA cycles in which PrP^{Sc} signal was detected up to the 3.1×10⁶-fold dilution of scrapie brain. Thereafter, this and all the successive dilutions in which no PrP^{Sc} signal was detected were diluted 10-fold into normal brain homogenate and subjected to a new round of 118 PMCA cycles. This second round of PMCA enabled detection of PrP^{Sc} up to the 5×10¹⁰-fold dilution of scrapie-infected hamster brain. By comparing the signal intensity of PrP^{Sc} with or without PMCA, the increase of sensitivity was around 10-million fold. This sensitivity can be further increased by performing more rounds of saPMCA.

### Example 5

### Infinite Prion Replication In Vitro by saPMCA

The principle behind saPMCA predicts that prions may be replicated indefinitively in vitro by successive dilutions and serial rounds of amplification. In order to evaluate this hypothesis, hamster brains infected with 263K scrapie were homogenized and diluted 10⁴-fold into a 10% normal hamster brain homogenate. Samples were either immediately frozen or subjected to 20 PMCA cycles. After this first round of PMCA, a small aliquot of the amplified and the frozen samples was taken and diluted 10-fold into more normal brain homogenate. These samples were again immediately frozen or amplified by 20 PMCA cycles. This procedure was repeated several times and PrP^{Sc} amplification was determined by Western blot after proteinase K (PK) digestion to remove remaining PrP^{C}. In further studies 17 rounds of PMCA were performed. In the final series of PMCA, the amount of scrapie brain homogenate is equivalent to a 10⁻²⁰ fold dilution. Estimation of the amount of PrP^{Sc} inoculum present indicates that, after this dilution, less than 1 molecule of brain-derived protein was present, whereas the amount of newly generated PrP^{Sc} corresponds to approximately 1×10¹² molecules, which is equivalent to the concentration of PrP^{Sc} present in a 100-fold dilution of scrapie brain. The amplified samples for the 10⁻²⁰ dilution were further diluted and subjected to several rounds of PMCA separated by 100-fold dilutions to reach a final dilution of scrapie brain homogenate equivalent to 10⁻⁴⁰. The serial replication of PrP^{Sc} was additionally continued up to a 10-55 dilution by performing a series of 1000-fold dilutions followed by 48 cycles of PMCA. The inventors conclude from these results that PMCA enables an infinite replication of PrP^{Sc} in vitro. Interestingly, the signal can be fully recovered even after 1000-fold dilution of the sample, suggesting that the amplification rate is at least 1000. Moreover, the rate of PrP replication was not altered upon dilution, which suggests that newly converted protein is capable of inducing PrP^{Sc} formation with a similar efficiency as brain-derived PrP^{Sc}. A control experiment in which the healthy brain homogenate was serially diluted into itself and subjected to the same number of PMCA cycles as described above but in the absence of PrP^{Sc} inoculum did not show any protease-resistant PrP under any condition.

### Example 6

### Reproducibility of Prion Detection by saPMCA

Reproducibility of amplification was measured by monitoring the PrP^{Sc} signal obtained before and after PMCA cycling under different experimental conditions. Equivalent samples containing a 10,000-fold dilution of scrapie brain into 10% healthy hamster brain homogenate were placed in distinct positions of the microplate sonicator and subjected to 48 PMCA cycles. Densitometric analysis of the PrP^{Sc} signal obtained in three different western blots of the same samples, show that although some small variability was observed, the differences were not statistically significant and could not be attributed to a position effect (rather, they were ascribed simply to experimental variability). To analyze further the reproducibility of the procedure, equivalent samples containing a 10,000-fold dilution of scrapie brain homogenate into 10% healthy hamster brain homogenate were subjected to 48 PMCA cycles in experiments done on different days. On 7 distinct days the amplification efficiency was virtually the same. Again, densitometric analysis showed that the signal was not statistically different in the distinct experiments. The influence of different, but equivalent inocula on the conversion efficiency was studied by amplifying preparations of 10,000-fold diluted scrapie brain homogenate obtained from 5 distinct hamsters into the same substrate. After 48 PMCA cycles, a large and similar conversion of PrP^{C} into PrP^{Sc} was observed. A similar result was obtained when normal brain homogenate from 5 different hamsters was used as a substrate for the amplification of a unique PrP^{Sc} inoculum. However, densitometric analysis of the experiments showed that in both cases one sample gave statistically significant different level of amplification than the other four samples. These results suggest that perhaps individual variability on the expression of PrP or conversion factors may lead to changes on the extent of prion conversion in vitro. In each of the studies described above PrP^{Sc} was not detectable in samples containing the same material but kept frozen without amplification.

### Example 7

Specificity of Prion Detection by saPMCA

Specificity of detection is very important for a diagnostic assay. Specificity of cyclic amplification was evaluated in a blind study in which 10 brain samples of scrapie-affected hamsters and 11 samples of healthy animals were subjected to 48 PMCA cycles and PrP^{Sc} was detected by Western blot analysis after proteinase K (PK) digestion. The results showed that, while 100% of the samples derived from sick animals were positive after PMCA, none of the samples coming from normal animals showed any PrP^{Sc} signal. Out of the 10 positive control samples, 7 corresponded to a 10,000-fold dilution of brain, 2 corresponded to a 50,000-fold dilution and 1 corresponded to a 100,000-fold dilution. None of these 10 samples showed any PrP^{Sc} signal in Western blot without PMCA amplification (data not shown). The interpretation of this data is that, under the conditions used, PMCA leads to 100% specificity in PrP^{Sc} detection.

As demonstrated before, the amplification rate using PMCA depends upon the number of incubation/sonication cycles carried out (Saborio et al., 2001). Thus, the inventors decided to evaluate whether a PrP^{Sc}-like signal might appear on negative samples after many PMCA cycles. For this purpose, a 10% healthy hamster brain homogenate in the absence (negative control) or in the presence (positive control) of an aliquot of a 50,000-fold diluted scrapie brain was subjected to 24, 48, 96, or 144 PMCA cycles and PrP^{Sc} signal detected by Western blot analysis. The results clearly indicated that PrP^{Sc} reactivity was detected only after PMCA in the positive control samples with an intensity that depended upon the number of cycles performed. In comparison, in the negative control samples, no PrP^{Sc} was ever detected, regardless the number of PMCA cycles carried out. In order to evaluate the relationship between the extent of PrP^{Sc} formation and the number of PMCA cycles, the inventors attempted to fit the data to a mathematical formula. Taking into account all the points available (again done by triplicate) the best fitting was obtained with a sigmoidal curve (Equation: signal intensity=1882/(1+e^{-(number of cycles-53.6)/21.8}), indicating that after an exponential relationship between the extent of conversion and the number of cycles, the formation of new PrP^{Sc} reaches a plateau. This plateau can be due to the exhaustion of all PrP^{C} substrate by conversion into PrP^{Sc} or to the lost of conversion efficacy by inactivation of the substrate or putative conversion factors after long times of incubation/sonication. This problem was resolved by saPMCA. When the data was fitted excluding the last time point, the best fit was obtained with an exponential curve (Equation: signal intensity=67/e^{0.98(number of cycles)}). These findings support the idea of an exponential dependence on the number of PMCA cycles when conversion conditions are not limiting (less than 100 cycles).

Specificity was further studied in an even more challenging situation in which several rounds of PMCA were done after diluting the material to refresh the substrate. Brains from healthy hamsters and from animals infected with 263K scrapie were diluted 10⁴-fold into a 10% normal hamster brain homogenate. Samples were subjected to 48 PMCA cycles. After this first round of PMCA, a small aliquot of the amplified samples was taken and diluted 10-fold into more normal brain homogenate. These samples were again amplified by 48 PMCA cycles. This procedure was repeated several times and PrP^{Sc} generation was determined by Western blot analysis after PK digestion. In this study 10 rounds of PMCA to reach a final dilution of the original brain equivalent to 10⁻¹³ led to continuous formation of PrP^{Sc} only when the initial inoculum was derived from scrapie-infected animals. No PrP^{Sc} was ever detected in the absence of PrP^{Sc} inoculum, indicating that, even after 480 PMCA cycles, the system retains high specificity and no false positive samples were observed.

### Example 8

### saPMCA Allows Detection of a Single Molecule of PrPSc

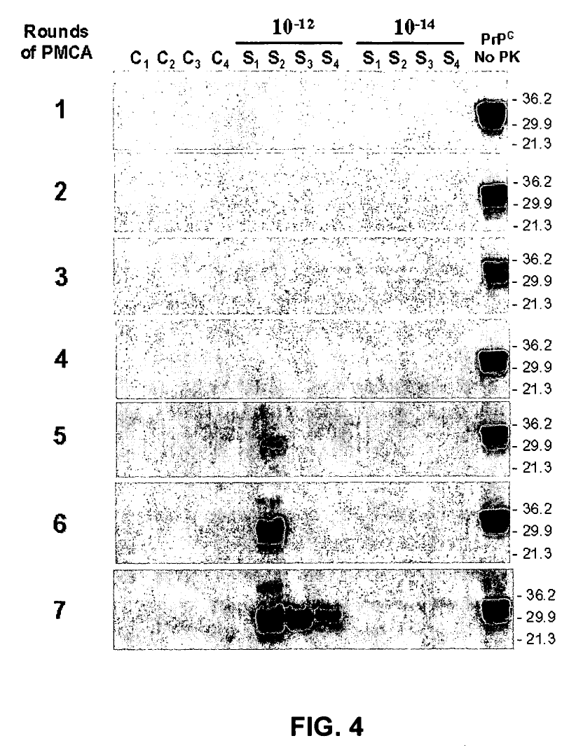

In order to estimate the minimum number of molecules of PrP^{Sc} that the inventive saPMCA can detect in a given sample, a scrapie brain homogenate was diluted 1×10⁻¹² fold into conversion buffer and subjected this material to saPMCA. According to inventors estimation, the PrP^{Sc} concentration in the scrapie infected brain used for these studies was approximately 67 ng/μl. This result indicates that a 1×10-12-fold dilution should contain ˜6.7×10⁻² g/μl or 1.3 molecules of PrP^{Sc} monomer per μl. Since in this study a volume of 20 μl was used, the sample tested contains approximately 26 molecules of monomeric PrP^{Sc}. Strikingly, after 5 rounds of saPMCA siganl was detected in one of the 4 replicates used and after 7 rounds of amplification, the inventors detected a signal in 3 of the 4 replicates (FIG. 4). Importantly, no amplified product was detected when a 10⁻¹⁴ fold dilution of brain was used (a sample that should contain no molecules of PrP^{Sc}) or in any of the control samples in which no PrP^{Sc} was present (FIG. 4). No signal was detected in a 10⁻¹³ fold dilution (data not shown).

Recent data have shown that the minimum size of the particle capable to sustain infectivity and induce the cell-free conversion of PrP^{C} into PrP^{Sc}, contains between 14 and 28 molecules of PrP monomers. Indicating that saPMCA can amplify a single particle of oligomeric infectious PrP^{Sc}. This unprecedented amplification efficiency is comparable only to the effectiveness of PCR amplification of DNA. Moreover, while at these levels of amplification, PCR often result in artifactual amplification products, the inventor have rarely seen a false positive using PMCA.

### Example 9

### Comparison of the Sensitivity of Prion Detection between saPMCA and Standard Tests used for Rapid Detection of BSE

As mentioned earlier, the serious consequences of the BSE epidemics and the increasing concern regarding the iatrogenic transmission of vCJD motivated the development of several biochemical methods to detect PrP^{Sc}. Five tests have been approved by the European community and are widely used in BSE surveillance in several countries (Moynagh and Schimmel, 1999; Soto, 2004). All these tests correspond to the immunological detection of PrP^{Sc} either by Western blot, ELISA, or CDI (for a review, see (Soto, 2004)). To compare the sensitivity of tests using these principles (adapted to detect hamster PrP^{Sc}) with PMCA detection, the inventors performed studies in parallel with different methods using the same samples, all prepared into 10% normal brain homogenate to facilitate the comparison (Table 3). Western blot is the most standard but least sensitive of these tests, allowing for the detection of a minimum of 4.0 ng of PrP^{Sc} in 20 μl of sample, which is equivalent to 8×10¹⁰ molecules of misfolded protein. In our hands, simple ELISA was 8-fold more sensitive than Western blotting. However, more sensitive ELISA tests have been reported, such as the two-sites immunometric sandwich ELISA (Deslys et al., 2001). According to literature estimations, CDI detects PrP^{Sc} up to a maximum dilution of scrapie brain homogenate equivalent to 2×10⁵, indicating that it is 27-fold more sensitive than Western blotting (Table 3). One strategy that has been used to enhance PrP^{Sc} detection is the specific precipitation and concentration of the protein using phosphotungstic acid (PTA) (Safar et al., 1998; Wadsworth et al., 2001). In our hands, PTA precipitation of PrP^{Sc} from scrapie brain led to a 50-fold increase in detection compared to standard Western blotting (Table 3). By comparison, one round of 100 PMCA cycles resulted in an average of 2500-fold more sensitive detection of PrP^{Sc} as compared with Western blotting. This sensitivity threshold indicates that one round of PMCA can detect as little as 3.2×10⁷ molecules of PrP^{Sc}. Strikingly, two rounds of PMCA were able to systematically detect PrP^{Sc} up to a maximum dilution of the scrapie brain equivalent to 2×10¹⁰, indicating a sensitivity of 6.5 million times higher than standard Western blotting (Table 3). In other words, two rounds of 100 PMCA cycles can detect as little as 12,300 molecules of PrP^{Sc}.

Seven successive rounds of PMCA cycles produce a signal after amplification even when the starting material was a 1×10⁻¹² fold dilution of sick brain homogenate. This amplification leads to an increase of sensitivity of 3-billion times respect to standard western blot (Table 3). Until now, the animal bioassay of infectivity was by far the most sensitive assay available for detection of prions. Among the animal bioassays, hamsters infected with the 263K scrapie strain are the most rapid and sensitive, because animals can be infected with the lowest quantity of infectious agent and disease symptoms are observed at the shortest time after inoculation. Indeed, a 1×10⁻⁹ dilution of sick brain is the minimum amount that can still produce disease in 50% of the animals (mean lethal dose or LD50). In our experiments the minimum dilution that produced disease in all animals was 4×10⁻⁹, indicating that the bioassay can detect as little as 107,000 molecules of misfolded protein, which represent a 725,000-fold higher sensitivity than Western blotting (Table 3). Remarkably, our findings with saPMCA using the same samples as for the infectivity studies demonstrate that two and seven rounds of saPMCA are >8- and >4000-times more sensitive than the most efficient animal bioassay, respectively (Table 3).

TABLE 3

| | | Minimum | Minimum | |
|---|---|---|---|---|
| | Maximum | PrP | number | |
| | dilution | quantity | of PrP | Increase in |
| Assay | detected^{a} | detected^{b} | molecules^{c} | sensitivity^{d} |
| Western blot | 3.0 × 10⁻³ | 4.0 ng | 8.0 × 10¹⁰ | 1 |
| ELISA | 3.7 × 10⁻⁴ | 0.5 ng | 1.0 × 10¹⁰ | 8 |
| Phosphotunstic | 6.0 × 10⁻⁵ | 80 pg | 1.6 × 10⁹ | 50 |
| acid | | | | |
| precipitation | | | | |
| Conformation | 5.0 × 10⁻⁵ | 67 pg | 1.3 × 10⁹ | 60 |
| dependent | | | | |
| immunoassay^{e} | | | | |
| Animal | 2.0 × 10⁻⁹ | 5.3 fg | 1.1 × 10⁵ | 725,000 |
| bioassay | | | | |
| One round | 1.2 × 10⁻⁶ | 1.6 pg | 3.2 × 10⁷ | 2,500 |
| PMCA^{f} | | | | |
| Two rounds | 5.0 × 10⁻¹⁰ | 0.7 fg | 1.3 × 10⁴ | 6,000,000 |
| PMCA | | | | |
| (saPMCA)^{f} | | | | |
| Seven rounds | 1.0 × 10⁻¹² | 1.3 ag | 26 | 3,000,000,000 |
| of PMCA^{f} | | | | |

^{a}The maximum dilution detected corresponds to the last dilution of 263K scrapie brain in which PrP^{Sc} is detectable.
^{b}The minimum quantity of PrP^{Sc} detectable in a brain sample volume of 20 μl.
^{c}The number of prP molecules detected in a 20 ml sample volume was estimated by comparison with recombinant PrP.
^{d}The increase of sensitivity is expressed in relation to the standard Western blot assay using 3F4 antibody.
^{e}The data for confomation-dependent immunoassay was taken from the literarure, whereas all the others werer experimentally caluclated..
^{f}The data for PCMA correspond to the average obtained in three different experiments using 100 PCMA cycles in each round.

### Example 10

### Prion Detection in Peripheral Tissues by saPMCA

The practical application of a prion diagnostic assay depends on the possibility of detecting PrP^{Sc} in peripheral tissues and biological fluids. Among the peripheral tissues that have consistently been shown to be infectious and to play a role in prion neuroinvasion are the lymphoid organs, and in particular the spleen (Aguzzi, 2003). Therefore, in order to evaluate the possibility to use PMCA to detect PrP^{Sc} in the periphery, groups of scrapie sick and normal animals were sacrificed, their spleen homogenized, mixed with normal hamster brain homogenate, and subjected to PMCA. These animals were inoculated intra-cerebrally with 263K hamster scrapie and sacrificed after clinical signs of the disease were clear. Before, amplification no detectable signal corresponding to PrP^{Sc} was observed in any of the animals. However, after 96 PMCA cycles, all the 10 samples coming from spleen of sick animals gave a clear signal corresponding to PrP^{Sc}, whereas no signal was detected in any of the 13 control samples. The extent of PrP^{Sc} signal was different among distinct samples, probably reflecting the variable quantity of PrP^{Sc} present in the spleen in different individuals.

### Example 11

### Prion Detection in Blood

Methods

Blood samples. The samples used for these studies were obtained from Syrian Golden hamsters inoculated intra-peritoneally with 100μl of a 10% brain homogenate from animals affected by 263K scrapie strain (or with vehicle) (Castilla et al., 2005a). At the indicated times (Table 4), several animals per group were sacrificed and blood was collected directly from the heart using a syringe containing EDTA. Blood was placed in tubes containing sodium citrate and separated in aliquots of 1 ml. Samples were processed to separate the buffy coat fraction (Castilla et al., 2005b).

PMCA Procedure. Buffy coat was subjected to freezing-thawing 3 times and centrifuged at 100,000×g for 1 h at 4° C. The pellet was resuspended in 100 ill of 10% normal brain homogenate. The preparation of the normal brain homogenate containing the PrP^{C} substrate having required conversion factors is to obtain a good efficiency of amplification. Healthy animals were perfused with phosphate-buffered saline (PBS) plus 5 mM EDTA prior to harvesting the tissue. Ten percent brain homogenates (w/v) were prepared in conversion buffer (PBS containing NaCl 150 mM, 1.0% Triton X-100 and the Complete Protease Inhibitor Cocktail (containing EDTA) from Roche, Switzerland) and samples clarified by a brief, low-speed centrifugation (1500 rpm for 30 s). Tubes containing the samples to be amplified were positioned on an adaptor placed on the plate holder of a microsonicator (Misonix Model 3000, Farmingdale, N.Y.) and programmed to perform cycles of 30 min incubation at 37° C. followed by a 20 sec pulse of sonication set at 60-80% potency (Castilla et al., 2005a). The microplate horn was kept in an incubator set at 37° C. during the whole process and thus the incubation was performed without shaking. A more detailed technical protocol for automated PMCA, including a troubleshooting section, has been described (Castilla et al., 2004; Saa et al., 2004).

PrP^{Sc} detection. The protease-resistant form of PrP was detected by western blots after digestion with proteinase K (50 μg/ml) for 60 min at 45° C. with agitation. The digestion was stopped by adding electrophoresis sample buffer. Proteins were fractionated by sodium dodecyl sulphate-polyacrylamide gel electrophoresis (SDS-PAGE), electroblotted into nitrocellulose membrane, and probed with 3F4 antibody (Signet, Dedham, Mass.) diluted 1:5,000 in PBS, 0.05% Tween-20. The immunoreactive bands were visualized by enhanced chemoluminesence assay (Amersham, Piscataway, N.J.). Western blots signals were analyzed by densitometry, using a UVP Bioimaging system EC3 apparatus (Upland, Calif.).

Results

The best source for routine diagnosis of infectious disease is a biological fluid, such as urine and blood. Compelling evidence indicate that the infectious agent is present in blood albeit in very small and, so far, biochemically undetectable amounts. In order to evaluate the application of saPMCA for detection of prions in blood, samples were taken from 18 hamsters with clinical signs of scrapie and 12 normal healthy controls. Buffy coat was extracted as described in methods and added to 10% normal hamster brain homogenate. After 144 PMCA cycles, 1 of the 18 scrapie samples showed a signal corresponding to amplified PrP^{Sc}. After a second round of 144 cycles PMCA, PrP^{Sc} was observed in 9 of the samples, but none of the control samples. After a total of 6 rounds of PMCA, 16 of the 18 scrapie samples gave a clear positive signal, whereas none of the 12 control samples showed any detectable signal. These results indicate that PMCA enable detection of prions in blood with 89% sensitivity and 100% specificity (no false positives). Thus far, the only assay capable of detecting prions in blood is the animal bioassay, which takes almost 2 years to lead to conclusive results. The sensitivity of the bioassay for prion detection in blood is around 31%, which corresponds to an average of different experiments reported by diverse investigators (Brown et al., 2001). Therefore, saPMCA has a dramatically higher sensitivity than even the most sensitive bioassay. Furthermore, since no other method can detect prions in blood, it is yet not clear whether or not all sick animals are expected to contain prions in their blood.

### Example 12

### Prion Detection in Blood of Pre-Symptomatic Animals

In order to evaluate the application of PMCA for detection of prions in blood during the pre-symptomatic phase, groups of hamsters were injected intraperitoneally (i.p.) with vehicle (phosphate buffered saline or PBS) or with 10% brain homogenate of 263K scrapie strain. At different times during the incubation period, groups of animals were sacrificed, blood collected and the buffy coat fraction separated as described previously (Castilla et al., 2005b). Samples were resuspended in healthy hamster brain homogenate and subjected to 144 PMCA cycles. To refresh the substrate, after a round of PMCA cycling samples were diluted 10-folds into normal brain homogenate and another round of 144 PMCA cycles was done. This procedure was repeated 7 times, because according to our results, 7 rounds of 144 PMCA cycles enable detection of 10-30 molecules of monomeric hamster PrP (unpublished observations), which in light of recent data (Silverira et al., 2005) corresponds to a single unit of infectious oligomeric PrP^{Sc}.

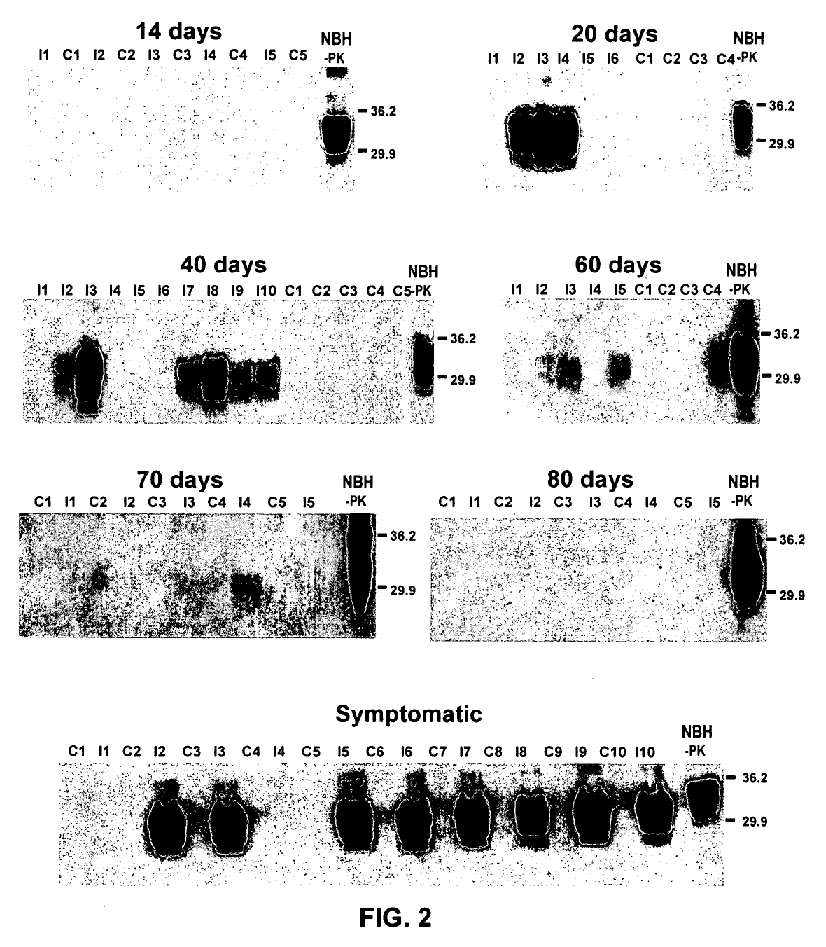

The first group of hamsters was sacrificed two weeks after i.p. inoculation. None of the 5 infected or control animals showed any detectable quantity of PrP^{Sc} in their blood (FIG. 2, Table 4). This result indicates that PrP^{Sc} present in the inoculum disappeared to undetectable levels during the first few days after inoculation. Interestingly, PrP^{Sc} was readily detectable in blood one week later, 20 days post-inoculation, in 50% of the animals infected, but in none of the controls (FIG. 2, Table 4). The highest percentage of positive animals during the pre-symptomatic phase was observed 40 days after i.p. inoculation, in which sensitivity of PrP^{Sc} detection was 60%. Surprisingly, detection of PrP^{Sc} in blood became harder after 60 days post-inoculation. Indeed, only I out of 5 animals scored positive at 70 days, whereas none of the 5 infected hamsters had detectable PrP^{Sc} in blood 80 days after inoculation (Table 4). At the symptomatic stage, which in this experiment-was at 114.2±5.6 days, 80% of animals had PrP^{Sc} in their blood (FIG. 2), confirming the earlier report in hamsters infected intra-cerebrally with 263K prions (Castilla et al., 2005b). Importantly, a false positive result was not detected in any of the 38 control samples analyzed (Table 4).

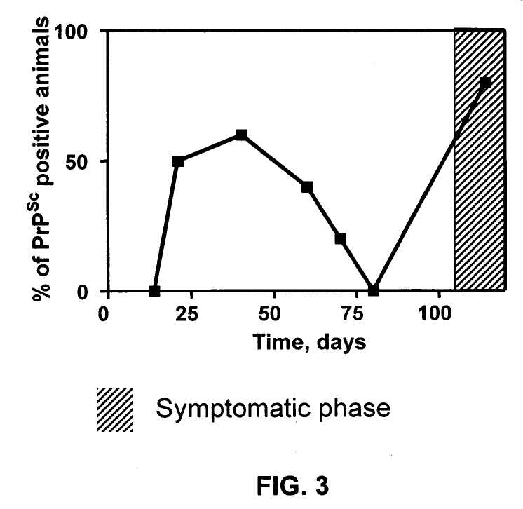

The distribution of PrP^{Sc} detection at different times of the incubation period, showed an interesting trend (FIG. 3). A first peak of PrP^{Sc} detection was observed early on during the pre-symptomatic phase, between 20-60 days post-inoculation. It has been reported that peripheral administration of prions results in an early phase of replication in lymphoid tissues and spleen, before any infectious material reaches the brain (Kimberlin and Walker, 1979; Glatzel and Aguzzi, 2000). Indeed, little or no infectivity can be detected in brain of animals peripherally inoculated during the first half of the incubation period. So, it is likely that the source of PrP^{Sc} in blood during the early pre-symptomatic phase is the spleen and other lymphoid organs. Surprisingly, the quantity of PrP^{Sc} in blood goes down after this initial phase and actually disappears 80 days post-inoculation (FIG. 3). The time window of no-detectability of PrP^{Sc} appears to coincide with the moment in which infectivity is migrating from the periphery to the brain. At the symptomatic period, the inventors were able to detect PrP^{Sc} in the blood of most of the animals (FIG. 3). According to published studies and the inventors experience with this model, large quantities of PrP^{Sc} appear in the brain only a few weeks before the onset of clinical signs (Kimberlin and Walker, 1986; Soto et al., 2005). Thus, PrP^{Sc} in blood samples at the symptomatic stage is likely coming from brain leakage.

It is well established by infectivity assays in animals that blood carries prions both in the symptomatic and pre-symptomatic stages of the disease (Brown et al., 2001; Brown, 2005; Hunter, 2002). Upon experimental BSE infection of sheep, infectivity was transmitted by blood transfusion from asymptomatic infected animals (Hunter, 2003), indicating that the infectious agent is present in blood during the incubation period. In humans, until recently there was no evidence of transmission of human TSEs by blood transfusion. However, recently three cases of vCJD have been associated to blood transfusion from asymptomatic donors who subsequently died from vCJD7 (Peden et al., 2004). The alarmingly high proportion of cases transmitted by blood transfusion suggests that prions exist in relatively elevated quantities in the blood of individuals silently incubating vCJD. Based on studies with animal models, it is believed that all the population may be susceptible to vCJD infection, although clinical cases have so far occurred only in methionine homozygotes at codon 129 in the human prion protein gene. Because the incubation period may be several decades, it is currently unknown how many people may be in an asymptomatic phase of vCJD infection. In addition, it is possible that some infected patients may never develop clinical symptoms but will remain asymptomatic carriers who can potentially transmit the disease to other individuals. In the absence of screening tests and effective therapies to treat this disease, a formidable worldwide public health challenge lies ahead to prevent further infections, to assess infection rates and to treat infected patients. The inventor's findings represent the first time in which PrP^{Sc} (the major component of infectious prions) has been detected biochemically in blood of infected but asymptomatic experimental animals. The PMCA technology has also been adapted to amplify prions from human origin (Soto et aL., 2005; unpublished results). The ability to detect accurately PrP^{Sc} in the pre-symptomatic stages of vCJD would be a major breakthrough with tremendous applications to reduce the risk that many more people get secondarily contaminated with this fatal and terrible disease.

TABLE 4
Number of animals used and results obtained on the
pre-symptomatic detection of PrP^{Sc} in blood.

| | Controls | Infected | Sensitivity/ |
|---|---|---|---|
| Time, days | Positives/Total | Positives/Total | specificity |
| 14 | 0/5 | 0/5 | 0%/100% |
| 20 | 0/4 | 3/6 | 50%/100% |
| 40 | 0/5 | 6/10 | 60%/100% |
| 60 | 0/4 | 2/5 | 40%/100% |
| 70 | 0/5 | 1/5 | 20%/100% |
| 80 | 0/5 | 0/5 | 0%/100% |
| Symptomatic | 0/10 | 8/10 | 80%/100% |

### Example 13

### Prion Amplification using Cellular Substrate

Traditionally PMCA amplification was done using brain homogenate from the same species as a source of PrPC and conversion factors. However, utilization of brain imposes practical and ethical problems, especially in the case of human samples. To overcome this difficulty the inventors have implemented neuroblastoma cells overexpressing normal prion protein as a substrate for conversion. The efficiency of amplification by saPMCA was found to be similar using brain homogenate or the neuroblastoma cell lysate.

### Example 14

### PrP^{Sc} Generated In Vitro by saPMCA is Biochemically and Structurally Identical to Brain-Derived PrP^{Sc}

saPMCA enable generation of PrP^{Sc} samples that do not contain any brain-derived PrP^{Sc}. This material is ideal for analyzing the biochemical and structural properties of the in vitro-produced protein and comparing them with the properties of in vivo-generated PrP^{Sc}. A first comparison using Western blot profiles indicates that in vitro replication leads to a protein with identical electrophoretic mobility and glycosylation pattern to the disease-associated misfolded protein. Indeed, experiments using PrP^{Sc} inoculum from different species/strains with distinct Western blot profiles showed that newly generated PrP^{Sc} always follow the pattern of the misfolded protein used as template (Soto et al., 2004). Furthermore, amino acid composition analysis of highly purified PrP^{Sc} produced in vitro shows very similar results to those found using brain-derived PrP^{Sc}, indicating that the cleavage site after proteinase K (PK) digestion is the same in both proteins. This is important because PrP^{Sc} from different strains has been shown to have a distinct PK cleavage site due to the different folding or aggregation of the protein (Chen et al., 2000; Collinge et al., 1996). The similar glycosylation pattern of newly-generated and brain-derived PrP^{Sc} was further confirmed in experiments in which the proteins were treated with endo-glycosidase. The results demonstrated that the enzymatic removal of glycosylated chains occurred with similar efficiency in both proteins and that the unglycosylated bands have the same molecular weight.

A typical feature of misfolded PrP that has been extensively used to distinguish it from the normal protein isoform is the high resistance of the pathological protein to protease degradation. To compare the protease resistance profile, similar quantities of PMCA-generated PrP^{Sc} (produced after a 10⁻²⁰ dilution of scrapie brain homogenate) and brain-derived PrP^{Sc} were treated for 60 min with 50, 100, 150, 200 and 250, 1000, 2500, 5000 and 10000 μg/ml of PK. Both proteins were highly resistant to these large PK concentrations, and, strikingly, the pattern of resistance was virtually identical. This result is very significant because protease resistance is one of the hallmark properties of disease-associated PrP, and its quantity correlates tightly with infectivity (McKinley et al., 1983). Several procedures have been reported to produce protease-resistant forms of PrP, but in most of these cases the protease resistance was only detected at low concentrations of the enzyme and was thus not comparable to the extent of protease-resistance seen in bona-fide PrP^{Sc} (Jackson et al., 1999; Lee and Eisenberg, 2003; Lehmann and Harris, 1996).

Another typical property of misfolded PrP is its high insolubility in non-ionic detergents. More than 95% of PrP^{Sc} derived both from brain and from PMCA was detected in the pellet after incubation and centrifugation in the presence of 10% sarkosyl, indicating that the two proteins are highly and similarly insoluble. Insolubility of PrP^{Sc} was lost when the proteins were treated with >2 M guanidinium hydrochloride, indicating that PrP^{Sc} from both origins was equally sensitive to denaturation by a chaotropic agent.

The main difference between PrP^{C} and PrP^{res}, which is responsible for the other biochemical distinctions, is the secondary structure of the two proteins; whereas PrP^{C} is mainly α-helical, PrP^{Sc} is rich in β-sheet conformation (Cohen and Prusiner, 1998; Pan et al., 1993). To study the secondary structure, PrP^{Sc} was highly purified from the brain of scrapie-sick hamsters or from samples amplified after a 10⁻²⁰ dilution. The standard purification procedure based on differential precipitation in detergents and protease degradation was used and purity was estimated to be >90% by silver staining after electrophoresis and by amino acid composition analysis. Structural studies conducted using Fourier Transform Infrared spectroscopy of in vitro-generated PrP^{Sc} showed a spectrum consisting of high levels of β-sheet content that was very similar to the spectrum obtained for purified brain-derived PrP^{Sc}. Deconvolution and fitting analysis of the spectra showed a virtually identical profile of secondary structures for both proteins, which are consistent with those previously reported for hamster PrP^{Sc} (Caughey et al., 1998; Pan et al., 1993). Importantly, the spectra showed a relatively small content of α-helical structure, as expected for disease-associated misfolded prion protein (May et al., 2004). The lack of α-helical structure is considered a drawback for most of the in vitro PrP refolding assays in which the PrP^{res}-like form is almost entirely organized in an aggregated β-sheet structure (May et al., 2004). The high levels of β-sheet structure as well as the presence of random coil and ax-helix for PMCA-generated PrP^{Sc} were also confirmed by circular dichroism studies. FTIR spectra of recombinant full-length hamster PrP^{C} produced in bacteria showed the expected high proportion of α-helix and random coil and a <10% of β-sheet structure.

The high content of β-sheet structure of PrP^{Sc} results in a high tendency to form larger order aggregates in vitro and in vivo (Ghetti et al., 1996; Prusiner et al., 1983). To study the ultrastructural characteristics of the aggregates, samples from highly purified brain-derived and PMCA-generated PrP^{Sc} were analyzed by electron microscopy after negative staining. Both proteins make typical prion rod-like structures which are 10 to 20 nm in diameter and 50 to 100 nm in length, as previously described (Prusiner et al., 1983; Wille et al., 2000).

A hallmark property of prions is their capability to sustain autocatalytic replication in vivo (Prusiner, 1998). Injection of brain extracts containing PrP^{Sc} into an animal can further direct the conversion of normal PrP^{C}, and the misfolded protein can in this way keep replicating across animals and generations (Prusiner, 1998). The results suggest that newly formed PrP^{Sc} is able to maintain replication in vitro even in the absence of brain-derived PrP^{res}. However, in order to analyze whether the efficiency of conversion is the same, the inventors compared the rate of PrP^{C} conversion induced by brain-derived and PMCA-produced PrP^{res}. For these experiments, aliquots of both samples containing a similar amount of PrP^{Sc} equivalent to a 100-fold dilution of scrapie brain homogenate were further diluted into normal brain homogenate and subjected to 20 amplification cycles. Both samples were able to convert high levels of PrP^{C} to produce a similar amount of PrP^{res}. The efficient conversion was lost under these conditions when the samples were diluted more that 160-fold (16,000-fold in total). This result indicates that an approximately 300-fold amplification rate was obtained for both brain and PMCA PrP^{Sc} using 20 amplification cycles. As described before, the rate of amplification depends upon the number of cycles performed (for example a >6500-fold amplification was obtained when samples were subjected to 140 cycles), but again this rate was similar regardless of whether PrP^{Sc} came from in vivo brain samples or from in vitro-produced protein.

Finally, the inventors studied whether the converting activity of in vitro-generated PrP^{Sc} is as resistant to denaturation as has been reported for brain PrP^{res}. Samples of brain-derived and in vitro-generated PrP^{Sc} were subjected to thermal denaturation by incubation at 100, 110, 120 and 140° C. for 1 hr. Thereafter, these samples were used to trigger PrP^{Sc} formation by diluting them into normal brain homogenate and performing 20 PMCA cycles. Generation of new PrP^{Sc} was not altered by previously heating the samples at 1000 or 110° C., but this activity was dramatically reduced by incubating PrP^{Sc} at 120° C. and completely abolished after heating at 140° C. Interestingly the heat resistance profile of both brain-derived and PMCA-produced PrP^{Sc} was very similar, further supporting the hypothesis that the two forms resemble each other.

### Example 15

### In Vitro Generated PrP^{Sc} by saPMCA is Infectious

One objective that has long been pursued is the in vitro production of prion infectious material by inducing the misfolding of PrP. Successful completion of this experiment is widely regarded as the final proof for the controversial protein-only hypothesis of prion propagation (Soto and Castilla, 2004). The serial replication of PrP^{Sc} in vitro by PMCA provides a perfect system to achieve this aim because, after many rounds of amplification following serial dilution of PrP^{Sc} inoculum, the inventors are able to produce a preparation of misfolded protein that is biochemically and structurally identical to brain-derived PrP^{Sc} but lacks any molecule of the initial scrapie-infected inoculum. To determine the infectious capability of in vitro-generated PrP^{res}, groups of wild-type Syrian hamsters were inoculated intracerebrally (i.c.) with samples generated by 6 or 16 rounds of serial PMCA separated by 10-fold dilutions. Since at the first PMCA the dilution of scrapie brain homogenate was 10⁻⁴, the final dilution of scrapie material in these groups corresponds to 10⁻⁹ and 10⁻¹⁹, respectively. An additional 10-fold dilution in phosphate-buffered saline was performed in all samples before inoculation. Despite the large dilution, the quantity of PrP^{Sc} was maintained constant after amplification. Detailed estimation by quantitative Western blot analysis indicated that the amount of PrP^{Sc} in these samples was similar to the quantity of PK-resistant protein present in a 10⁻⁴ dilution of 263K hamster brain, which contains approximately 10¹⁰ molecules of the misfolded protein. From this amount the animals infected with the 10⁻¹⁰ dilution (group 6 in Table 5), 99.99999% of the material corresponded to newly generated PrP^{Sc} (0.99999×10¹⁰ molecules) and only 0.000001% to brain-derived PrP^{Sc} (1×10⁴ molecules). In the 10⁻²⁰ dilution (group 7), 100% of PrP^{Sc} was newly generated protein (1×10¹⁰ molecules). Strikingly, all the animals in these two sets (groups 6 and 7) showed typical signs of scrapie and died of the disease at around 170 days after inoculation (Table 5).

TABLE 5

| | Scrapie | Molecules of | Predicted survival | Observed survival |
|---|---|---|---|---|
| Group # | brain | PrP^{res(a)} | time^{b} | time^{c} (sick/total |
| (Amplified) | dilution | (Brain/PMCA) | (% sick animals) | animals) |
| 1 | 10⁻¹⁰ | ˜10⁴ | >600 days | >400 days |
| (None) | | (10⁴/0) | (0%) | (0/6) |
| 2 | 10⁻²⁰ | 0 | >600 days | >400 days |
| (Amplified*) | | (0/0) | (0%) | (0/6) |
| 3 | 10⁻¹⁰ | ˜10⁴ | >600 days | >400 days |
| (Amplified*) | | (10⁴/0) | (0%) | (0/6) |
| 4 | 10⁻²⁰ | 0 | >600 days | >400 days |
| (None) | | (0/0) | (0%) | (0/6) |
| 5 | 10⁻⁴ | ˜10¹⁰ | 94 +/− 2.7 days | 106 +/− 2.9 |
| (None) | | (10¹⁰/0) | (100%) | (6/6) |
| 6 | 10⁻¹⁰ | ˜10¹⁰ | N/A | 177 +/− 7.3 |
| (Amplified) | | (10⁴/0.99 × 10¹⁰) | | (6/6) |
| 7 | 10⁻²⁰ | ˜10¹⁰ | N/A | 162 +/− 3.5 |
| (Amplified) | | (0/10¹⁰) | | (6/6) |

^{a}The number of molecules of PrP^{res} were estimated based on quantitative Western blot, using known concentrations of recombinant Hampster PrP as a standard
^{b}The prediction of a survival times are based on previous data and published observations.
^{c}Observed survival time is expressed as average +/− standard error.
*indicates that the amplification was carried-out in PrP null brain homogenate.

Based on experience with the 263K experimental model and literature reports, the 10⁻⁹ dilution of scrapie brain homogenate is the last dilution in which infectivity is observed (and only in some animals). Therefore, the inventors hypothesized that dilutions equivalent to 10⁻¹⁰ and 10⁻²⁰ would not produce any detectable disease (Table 5). This estimation was confirmed by the results obtained in our control groups (Table 5; groups 2, 3 and 4). Two different negative control groups were done; the first one contained 10⁻¹⁰ and 10⁻²⁰ dilutions of the scrapie brain homogenate into normal hamster brain homogenate done in serial 10-fold dilutions in parallel to the samples for PMCA, but kept frozen without amplification (groups 1 and 2). The second control consisted of the scrapie brain homogenate diluted serially into PrP knockout mouse brain homogenate up to 10⁻¹⁰ and 10⁻²⁰ fold dilutions and subjected to the PMCA cycling (groups 3 and 4) in the same way as the study samples. None of the 6 animals in these four groups of negative control samples have yet shown any signs of disease up to 300 days after infection (Table 3). This result clearly indicates that infectivity seen in the PMCA amplified samples is associated with newly in vitro-generated PrP^{res}.

To compare the infectious capacity of PMCA-produced PrP^{Sc} with brain-derived infectivity, a group of animals were inoculated with a sample containing a similar amount of PrP^{Sc} as the one produced after 6 and 16 serial PMCA rounds. As mentioned above, careful estimation using Western blot analysis showed that the quantity of PrP^{Sc} after the serial PMCA assays was equivalent to a 10⁻³ dilution of scrapie brain homogenate (10⁻⁴ considering the further 10-fold dilution prior inoculation). A positive control group of animals (group 5) injected with this dilution of scrapie brain developed the disease with a mean survival time of 106 days (Table 5). The material for this experiment and the dilution used correspond exactly to the sample utilized to begin PMCA amplification, so it serves as the double control of the infectivity present in the sample prior to any dilution and amplification as well as a control for the infectivity associated to this amount of PrP^{res}. The survival time was shorter that the one obtained with the equivalent quantity of PMCA-generated PrP^{res}, indicating that the in vitro-generated misfolded protein was significantly less infectious. Infectivity titration studies can be done to find out exactly how much lower infectivity the inventors have in the samples, but based on the survival time, in vitro-generated PrP^{Sc} seem to be between 10 to 100 times less infectious than the same quantity of brain-derived PrP^{res}. The inventors are also performing a second passage of the infectious agent and preliminary results indicate that animals infected with material originally derived from PMCA are coming down with the disease similarly as animals injected with brain infectious material. These results indicate that the infectious agent generated in vitro is stable over time.

The clinical signs observed in the disease produced by the amplified samples were identical to those of the animals inoculated with infectious brain material and included hyperactivity, motor impairment, head wobbling, muscle weakness, and weight loss. In order to evaluate whether the biochemical and neuropathological characteristics of the disease were also the same, the inventors conducted a comparative study of the brains of animals affected by the disease induced by brain-derived PrP^{Sc} (group 5) and PMCA-generated PrP^{Sc} (groups 6 and 7). Brain samples from all the animals in these four groups contained a large and similar quantity of PrP^{res}, which has an identical glycosylation profile. Conversely, no protease-resistant protein was detected in the brain of negative control animals. To further evaluate whether or not PMCA-generated infectivity represents a new strain, the inventors compared the electrophoretic mobility after PK treatment and the glycoform pattern of PrP^{Sc} with those of two other standard scrapie strains in hamsters, namely 263K and drowsy. Whereas the western blot pattern of the PMCA generated PrP^{Sc} is identical to 263K (the strain used to produce new PrP^{Sc} by PMCA), it is substantially different from drowsy, a strain known to differ biochemically from 263K.

Histological analysis showed typical spongiform degeneration of the brain and samples from animals infected with in vitro-produced PrP^{Sc} showed a pattern and extent of vacuolation that was indistinguishable from those coming from the brains of hamsters inoculated with infectious brain material. The same similarities were also seen when tissue samples were stained for PrP accumulation and astrogliosis. Thus, based in all the biochemical, histological, and clinical analyses of the animals, the inventors concluded that in vitro-generated PrP^{Sc} triggers a similar neurological disorder as brain-derived PrP^{res}.

The data in examples 13 and 14 demonstrate that PrP^{Sc} generated in vitro by saPMCA is identical to the misfolded protein produced in the brain during the course of the disease. Although this is not needed for the practical application of saPMCA for TSE diagnosis, it shows the relevance of the assay in reproducing the disease process. These findings coupled with the automation, sensitivity, reproducibility and high throughput of the technology indicate that saPMCA might be a very useful assay for identification of compounds for TSE therapy.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. Aspects of one embodiment may be applied to other embodiments and vice versa. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,196,265
U.S. Pat. No. 5,567,595
U.S. Pat. No. 5,688,648
U.S. Pat. No. 5,863,727
U.S. Pat. No. 5,914,273
U.S. Pat. No. 6,008,373
U.S. Pat. No. 6,150,107
U.S. Pat. No. 6,562,209
U.S. Pat. No. 6,855,503
WO 00/13026
WO 01/19841
WO 02/04954
   GenBank Accession Numbers:
AAP84097
AAU02120
AAU02123
AAU93884
AY335912
AY723289
AY723292
AY748455
NM_{—}183079
NM_{—}011170
NP_{—}035300
NP_{—}898902
   Non Patent Publications:
Editorial, Nature 409:649, 2001
Aguzzi, Adv. Immunol., 81:123-171, 2003.
Aguzzi, Lancet., 349:742-743, 1997.
Baldwin et al., J. Biol. Chem., 270:19197-19200, 1995.
Balter, Science, 294:770-771, 2001.
Bieschke et al., Proc. Natl. Acad. Sci. USA, 101: 12207-12211, 2004.
Bird, Stat. Methods Med. Res., 12:261-278, 2003.
Boellaard et al., Clin. Neuropathol., 18:271-285, 1999.
Brown and Sassoon, Mol. Biotechnol., 22:165-178, 2002.
Brown et al., J. Lab Clin. Med., 137:5-13, 2001.
Brown et al., Transfusion, 38:810-816, 1998.
Brown et al., Transfusion, 39:1169-1178, 1999.
Brown, Vox Sang. 89, 63-70, 2005.
Bruce, Nat. Med., 6:258-259, 2000.
Butler, Nature, 395:205-206, 1998.
Castilla et al., In: Methods and Tools in Bioscience and Medicine. Techniques in Prion Research, Lehmann and Grassi (Eds.), Switzerland: Birkhauser Verlag, 198-213, 2004.
Castilla et al., Nat. Med. 11, 982-985, 2005b.
Castilla et al., Cell 121, 195-206, 2005a.
Caughey et al., J. Biol. Chem., 273:32230-32235, 1998.
Chen et al., Arch. Virol. Suppl., 209-216, 2000.
Cohen and Prusiner, Annu. Rev. Biochem., 67:793-819, 1998.
Collinge et al., Nature, 383:685-690, 1996.
Collinge, Annu. Rev. Neurosci., 24:519-550, 2001.
Collinge, Lancet., 354:317-323, 1999.
Collins et al., J. Clin. Neurosci., 7:195-202, 2000.
Cortelli et al., J. Sleep Res., 8(1):23-29, 1999.
Coulthard et al., Br. J. Radiol., 72:742-748, 1999.
Deslys et al., Nature, 409:476-478, 2001.
Ghetti et al., Brain Pathol., 5:61-75, 1995.
Ghetti et al., Brain Pathol., 6:127-145, 1996.
Glatzel and Aguzzi, Microbes. Infect. 2, 613-619, 2000.
Harlow and Lane, “Antibodies: A Laboratory Manual,” Cold Spring Harbor Laboratory, 1988
Heim and Wilesmith, Arch. Virol. Suppl., 127-133, 2000.
Henry and Knight, Rev. Med. Virol., 12:143-150, 2002.
Hill et al., Lancet., 349:99-100, 1997.
Hill et al., Lancet., 353:183-189, 1999.
Houston et al., Lancet., 356:999-1000, 2000.
Hunter et al., J. Gen. Virol., 83:2897-2905, 2002.
Hunter, Br. Med. Bull. 66, 171-183, 2003.
Ingrosso et al., Trends Mol. Med., 8:273-280, 2002.
Jackson et al., Science, 283:1935-1937, 1999.
Johnson and Gibbs, N. Eng. J Med., 339:1994-2004, 1998.
Kascsak et al., J. Virol., 61:3688-3693, 1987.
Kawashima et al., Lancet., 350:68-69, 1997.
Kimberlin and Walker, J. Comp Pathol. 89, 551-562, 1979.
Kimberlin and Walker, J. Gen. Virol. 67, 255-263, 1986.
Klohn et al., Proc. Natl. Acad. Sci. USA, 100(20):11666-11671, 2003.
Kocisko et al., Nature, 370:471-474, 1994.
Kordek, Folia Neuropathol., 38:151-160, 2000.
Lee and Eisenberg, Nat. Struct. Biol., 10:725-730, 2003.
Lehmann and Harris, Proc. Natl. Acad. Sci. USA, 93:5610-5614, 1996.
Llewelyn et al., Lancet., 363:417-421, 2004.
May et al., Trends Biochem. Sci. 29:162-165, 2004.
McKinley et al., Cell, 35:57-62, 1983.
Moynagh and Schimmel, Nature, 400:105, 1999.
Pan et al., Proc. Natl. Acad. Sci. USA, 90:10962-10966, 1993.
Peden et al., Lancet 364, 527-529, 2004.
Prusiner et al., Cell, 35:349-358, 1983.
Prusiner, In: Prions, Fields Virology, 4^{th} Ed., Knipe and Howley (Eds.), Lippincott Williams and Wilkens, PA, 3063-3087, 2001.
Prusiner, Proc. Natl. Acad. Sci. USA, 95:13363-13383, 1998.
Prusiner, Science, 252:1515-1522, 1991.
Saa et al., In: Amyloid Proteins: Methods and Protocols, Sigurdsson (Ed.), Humana Press, 53-65, 2004.
Saborio et al., Nature, 411:810-813, 2001.
Safar et al., Nat. Med., 4:1157-1165, 1998.
Scott et al., Proc. Natl. Acad. Sci. USA, 96:15137-15142, 1999.
Silveira et al., Nature 437, 257-261, 2005.
Soto and Castilla, Nature Med., 10:S63-S67, 2004.
Soto, Nature Rev. Microbiol., 2:809-819, 2004.
Soto et al., FEBS Lett. 579, 638-642, 2005.
Stahl et al., Biochemistry, 32:1991-2002, 1993.
Wadsworth et al., Lancet., 358:171-180, 2001.
Weber et al., Biomed. Pharmacother., 51:381-387, 1997.
WHO Report, 1998
Will et al., Lancet., 347:921-925, 1996.
Wille et al., J. Struct. Biol., 130:323-338, 2000.

1. A method for detecting a prion in a sample comprising:
(a) mixing the sample with non-pathogenic protein to make a reaction mix;
(b) performing primary amplification comprising;
(i) incubating the reaction mix;
(ii) disrupting the reaction mix;
(iii) repeating steps (i) and (ii) one or more times;
(c) performing serial amplification comprising;
(i) removing a portion of the reaction mix and incubating it with additional non-pathogenic protein;
(d) using an assay to detect prions in the reaction mix.

2. The method of claim 1, wherein the prion comprises mammalian PrP.

3. The method of claim 1, wherien the non-pathogenic protein comprises a detectable label.

4. The method of claim 1, further comprising incubating the sample at about 25 to 50° C.

5. The method of claim 1, further comprising incubating the sample for about 1 minute to about 10 hours.

6. The method of claim 1, wherein disrupting the sample is by sonication.

7. The method of claim 6, wherein the sonicator is programmable for automated operation.

8. The method of claim 6, wherein the sample does not directly contact the sonicator.

9. The method of claim 1, wherein the samples are sealed to prevent evaporation.

10. The method of claim 1, wherein steps (b)(i) and (b)(ii) are repeated 1 to 200 times.

11. The method of claim 1, wherein the reaction mixture further comprises a metal chelator.

12. The method of claim 11, wherein the metal chelator is EDTA.

13. The method of claim 1, wherein the non-pathogenic protein is from a cell lysate.

14. The method of claim 13, wherein the cell lysate is from cells over expressing PrP.

15. The method of claim 13, wherein the cell lysate is from cells expressing a mutant or a labeled PrP.

16. The method of claim 13, wherein the cell lysate is a brain homogenate.

17. The method of claim 13, wherein the brain homogenate is a mammalian brain homogenate.

18. The method of claim 13, wherein the source of the brain homogenate is the same species as the source of the sample.

19. The method of claim 1, wherein step (b) is performed over a period of about three days.

20. The method of claim 1, wherein the sample is a tissue sample from an animal.

21. The method of claim 20, wherein the tissue sample is from brain.

22. The method of claim 20, wherein the sample is from a peripheral organ.

23. The method of claim 22, wherein the peripheral organ is blood, tonsils, spleen or other lymphoid organs.

24. The method of claim 1, wherein the assay to detect prion is Western blot, animal bioassay, ELISA or CDI, cellular infectivity assay or a spectroscopic assay.

25. The method of claim 24, wherein the ELISA assay is a two-site immunometric sandwich ELISA.

26. A method for detecting a prion in a sample comprising;
(a) mixing the sample with non-pathogenic protein to make a reaction mix;
(b) performing primary amplification comprising;
(i) incubating the reaction mix;
(ii) disrupting the reaction mix;
(iii) repeating steps (i) and (ii) one or more times;
(c) performing serial amplification comprising;
(i) removing a portion of the reaction mix and incubating it with additional non-pathogenic protein;
(ii) repeating step (b);
(d) using an assay to detect prions in the reaction mix.

27. The method of claim 26 further comprising repeating step (c) one or more times.

28. The method of claim 27, wherein prion can be detected in a sample containing 2×10⁵ prion molecules or less.

29. The method of claim 27 further comprising inactivating residual prion.

30. A method to diagnose a disease in an animal comprising detecting the presence of a prion in a sample from the animal by the method of claim 1.

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. A kit for detection of prion in a sample comprising:
(a) non-pathogenic protein and at least one of the following:
(i) conversion buffer with a metal chelator, or
(ii) prion conversion factors.

45. The kit of claim 44, wherein the non-pathogenic protein is lyophylized.

46. The kit of claim 44, further comprising one or more of the following:
(a) conversion buffer,
(b) decontamination solution,
(c) a positive control,
(d) a negative control, or
(e) reagents for the detection of prion.

47. The kit of claim 44, wherein the non-pathogenic protein comprises a detectable label.

48. The kit of claim 44, further comprising reagents for labeling the non-pathogenic protein.

49. The kit of claim 46, wherein the reagents for detection of prion further comprise antibodies.
